(19) United States
(12) Patent Application Publication
(10) Pub. No.: US 2020/0226481 A1
(43) Pub. Date: Jul. 16, 2020

(54) METHODS AND SYSTEMS FOR MANAGING MEDICAL INFORMATION

(71) Applicant: 5 Health Inc., San Francisco, CA (US)

(72) Inventors: Yan Chuan SIM, Singapore (SG); Dorothea Li Feng KOH, Singapore (SG); Md Ihtimam Hossain BHUIYAN, Singapore (SG)

(21) Appl. No.: 16/742,466

(22) Filed: Jan. 14, 2020

Related U.S. Application Data
(60) Provisional application No. 62/792,171, filed on Jan. 14, 2019.
(60) Provisional application No. 62/886,242, filed on Aug. 13, 2019.

(51) Int. Cl. G06N 5/04; G06N 20/00; G16H 10/60; G16H 70/20; G06F 16/9535; G06F 40/40; G06F 3/0484; H04L 12/58

(52) U.S. Cl. CPC G06N 5/04; G06N 20/00; G16H 10/60; G16H 70/20; H04L 51/02; G06F 40/40; G06F 3/0484; H04L 51/046; G06F 16/9535

(57) ABSTRACT

In some variations, methods for managing medical information may include receiving through a user interface on a user computing device a user selection of medical content and a user selection of a tag to be associated with the medical content, and modifying a machine learning associations model based on the medical content and tag, wherein the machine learning associations model predicts queried medical content based on user input received through the conversation simulator. In some variations, methods for managing medical information may include receiving a medical content record specific to a user group, receiving at least one tag to be associated with the medical content record, and modifying a machine learning associations model based on the medical content record and the at least one tag, wherein the machine learning associations model predicts queried medical content based on user input received through a user interface.

### CROSS-REFERENCE TO RELATED APPLICATIONS

This application claims priority to U.S. Provisional Application Ser. No. 62/792,171 filed Jan. 14, 2019, and U.S. Provisional Application Ser. No. 62/886,242 filed Aug. 13, 2019, each of which is hereby incorporated herein by reference in its entirety.

### TECHNICAL FIELD

This invention relates generally to the field of medical treatment, and more specifically to managing medical information.

### BACKGROUND

The digitization and globalization of medical and scientific discoveries on disease origins, symptoms, and treatments have led to a knowledge explosion in medicine. Healthcare professionals have a tremendous amount of medical information that they have to learn, recall, and keep up to date on, in order to ensure that they are treating their patients with the latest standards of care. Healthcare professionals may be able to provide more effective medical treatment to patients if they are able to consistently and easily access such clinical and/or other medical information.

Conventional medical resources include hard copy printed resources (e.g., books, paper-based patient medical records). Printed resources are not easily or reliably updateable, and information that is no longer accurate may detract from proper medical treatment. Furthermore, there may be limited access to printed resources because they are difficult to share among multiple users. Some other medical resources are digital or electronics-based (e.g. hospital intranet or information systems), but tend to be time-consuming and/or difficult to navigate to obtain desired information, which may lead to unnecessary and harmful delays in providing medical treatment to patients.

Existing technologies such as internet search engines and medical knowledge databases can help a user search for specific information, but such resources are typically discrete, such that healthcare professionals must separately consult various databases to obtain the information they seek. These technologies will prove unscalable and even less tenable in the future, as the advancement in medical science continues to accelerate in building an ever-increasing volume of information. Furthermore, much of medical knowledge and information useful to a healthcare professional comes from a variety of online and offline sources including journals, textbooks, guidelines, websites, the hospital's own institutional guidelines and protocols, and even through the peer-to-peer sharing of information (e.g., clinical expertise, clinical practices that are cultural- and/or geographical-specific).

Thus, there is a need for new and improved systems and methods for generating more efficient and user-friendly access to medical resources for healthcare professionals.

### SUMMARY

In some aspects of the methods and systems described herein, a user may engage in chat conversations within an artificial intelligence environment, such as with an artificial intelligence medical assistant (e.g., represented by a chatbot or other conversation simulator) and/or one or more other users. The artificial intelligence medical assistant may provide medical information to one or more users in response to user inputs (e.g., queries) within a chat conversation. Additionally, media such as images or videos, or other attachments such as links, document files (e.g. files in ADOBE Portable Document Format (PDF) including guidelines and/or other information, spreadsheets, text or word documents, etc.) and/or clinical tools such as medical calculators may be shared among users and/or the artificial intelligence medical assistant. Furthermore, a user may create notes (e.g., associated with a patient) such as through text entry, dictation, and/or adding photos, videos or other combinations of media. Various medical information in the chats and/or notes may be generated and/or stored in new and/or existing electronic medical records associated with patients.

Generally, a method may include receiving through a user interface on a user computing device a user selection of medical content and a user selection of a tag to be associated with the medical content, and modifying a machine learning associations model based on the medical content and tag. The machine learning associations model may predict queried medical content based on user input received through the user interface. In some variations, the method may further include indexing the medical content and the tag for storage in one or more memory devices. The user interface may include a conversation simulator, which may be associated with a natural language processing model.

Furthermore, in some variations, the method may further include receiving a user input from at least one user through the user interface, predicting queried medical content associated with the user input based on the machine learning associations model, and displaying the predicted medical content on the user interface.

Various kinds of medical content and other content may be tagged and retrieved for display. For example, the content may include content displayed in the conversation simulator, such as text, an image, and/or a video. As another example, the content may include content displayed in an internet browser (e.g., in a mobile application associated with the artificial intelligence medical assistant on the user computing device, or in another browser mobile application on the user computing device) or in a document viewer.

In some variations, the method may incorporate user behavior by automatically prompting the user to make the user selection of medical content and the user selection of a tag associated with the medical content, based at least in part on user behavior. For example, a user communicating with a chat message exceeding a predetermined length may prompt the user to tag content in the chat message.

Furthermore, generally, a system may include one or more processors configured to display a user interface on a user computing device, receive through the user interface a user selection of medical content and a user selection of a tag to be associated with the medical content, and modify a machine learning associations model based on the medical content and tag, wherein the machine learning associations model predicts queried medical content based on user input received through the user interface. The one or more processors may be further configured to perform other aspects of the method described herein.

In some variations, a method may include receiving a medical content record specific to a user group, receiving at least one tag to be associated with the medical content record, and modifying a machine learning associations model based on the medical content record and the at least one tag, wherein the machine learning associations model predicts queried medical content based on user input received through a user interface. For example, the user group may be associated with a medical institution, organization, or other suitable group. The medical content may include content such as one or more of an call roster or schedule (e.g., on-call roster, inpatient roster, referral roster, etc.), drug formulary, medical practitioner directory, medical guidelines, and/or medical protocols. Such medical content may be specific to the associated medical institution. The medical content may include text, images, videos, and/or other suitable formats. In some variations, the method may further include indexing the medical content record and the at least one tag for storage in one or more memory devices. Furthermore, the method may include automatically providing one or more suggested tags to be associated with the medical content record. The one or more suggested tags may be based, for example, on the at least one received tag, such as according to the machine learning associations model. In some variations, the user interface may include a conversation simulator. The method may include predicting queried medical content associated with a user input based on the machine learning associations model.

Furthermore, generally, a system may include one or more processors configured to receive a medical content record specific to a user group, receive at least one tag to be associated with the medical content record, and modify a machine learning associations model based on the medical content record and the at least one tag, wherein the machine learning associations model predicts queried medical content based on user input received through a user interface.

Generally, a method may include receiving a user input at a user interface on a first computing device, wherein the user interface includes a conversation simulator, generating an authentication code in response to the user input, associating the authentication code with a user account at least in part by using a second computing device, and in response to associating the authentication code with the user account, providing access to the user account through the user interface at the first computing device. The conversation simulator may be associated with a natural language processing model. The user interface on the first computing device may, for example, include a web browser.

In some variations, providing access to the user account may include providing access to medical content associated with the user account. For example, access may involve allowing search of the medical content associated with the user account through the conversation simulator. Such medical content may, for example, include text, image, video, combinations thereof, and/or other suitable content.

A second computing device may be used to associate the authentication code with a user account in various manners. For example, in some variations, the second computing device may be associated with the user account and associating the authentication code with the user account may include providing the authentication code at the first computing device and determining that the authentication code is received by the second computing device. For example, the first computing device may be a desktop or laptop computer providing a web browser user interface, which may display an authentication code in the form of a scannable code (e.g., quick response (QR) code). The authentication code may be received at a mobile computing device (or other second computing device) and subsequently associated with a user account to enable access to the user account at the first computing device.

As another example, in some variations, the second computing device may be associated with the user account and associating the authentication code with the user account may include providing the authentication code to the second computing device, and determining that the authentication code is received by the first computing device. For example, the first computing device may be a desktop or laptop computer providing a web browser user interface. An authentication code such as a text-based code (e.g., delivered through SMS) may be provided to a mobile computing device (or other second computing device) and subsequently associated with a user account when provided to the first computing device, to enable access to the user account at the first computing device.

Generally, in some variations, a method may include, at one or more processors, identifying a medical content application module of interest, customizing the medical content application module based on a medical content record specific to a user group (e.g., a medical institution such as a hospital or other entity), and providing the customized medical content application module to a user associated with the user group, where the customized medical content application module may be provided through a user interface on a computing device, and where the user interface comprises a conversation simulator. The customized medical content application module may, for example, be displayed on the user interface to the user through the conversation simulator.

In some variations, providing the customized medical content application module may include accessing a stored customized medical content application module. For example, the selection of a medical content application module may be performed by an administrator associated with the user group (e.g., using a content management platform described in further detail below). Additionally or alternatively, customizing the selected medical content application module may be performed in real-time (or substantially in real-time) in response to a user input provided through the user interface, such as from a clinician. Such a customized medical content application module may be configured to provide medical content specific to the user group. The medical content record may, for example, include drug information, call roster or schedule, medical practitioner directory information, inventory information, pricing information, medical guidelines, a medical protocol, medical procedure code, billing and/or reimbursement and coding information, a dosing regimen, and/or the like.

Generally, in some variations, a system may include one or more processors configured to identify a medical content application module of interest, customize the selected medical content application module based on a medical content record specific to a user group, and provide access to the customized medical content application module to a user associated with the user group, in response to a user input at a user interface on a computing device, where the user interface comprises a conversation simulator.

### BRIEF DESCRIPTION OF THE DRAWINGS

FIG. 1 is a schematic illustration of an exemplary architecture for an artificial intelligence (AI) environment.

FIG. 2 is a schematic illustration of an exemplary variation of a user computing device.

FIG. 3 is a schematic illustration of an exemplary variation of an artificial intelligence medical assistant system.

FIG. 4A is an illustrative flowchart depicting an exemplary interaction between an artificial intelligence medical assistant system and a user computing device.

FIG. 4B is an illustrative flowchart depicting an exemplary variation of a method for predicting user intent and determining medical content in an artificial intelligence environment.

FIG. 4C is an illustrative flowchart depicting an exemplary variation of a method for incorporating feedback to update a model for predicting user intent and determining medical content in an artificial intelligence environment.

FIG. 5 is a schematic illustration of an exemplary variation of an AI environment.

FIG. 6 is an illustrative flowchart depicting an exemplary interaction between an artificial intelligence medical assistant system and a user computing device.

FIG. 7 is an illustrative flowchart depicting another exemplary interaction between an artificial intelligence medical assistant system and a user computing device.

FIGS. 8A-8D are exemplary variations of a GUI relating to a tutorial for training aspects of an artificial intelligence system.

FIGS. 9A-9D are exemplary variations of GUIs relating to tagging content in a chat conversation.

FIGS. 10A and 10B are exemplary variations of GUIs relating to tagging content in a document viewer.

FIGS. 11A and 11B are exemplary variations of GUIs relating to tagging content in a internet browser.

FIGS. 12A and 12B are exemplary variations of GUIs relating to tagging files in a chat conversation.

FIGS. 13A and 13B are exemplary variations of GUIs relating to an automatic prompt to a user to tag content.

FIG. 14 is an exemplary variation of a GUI relating to one method for a user to access previously tagged content.

FIG. 15 depicts an exemplary variation of a method for training a machine learning model within an AI environment.

FIG. 16 is a schematic illustration of an exemplary variation of part of a content management platform operable within an AI environment.

FIG. 17A is an exemplary variation of a GUI relating to management of clinic modules in a content management platform.

FIGS. 17B-17D are exemplary variations of datasheets associated with clinic modules in a content management platform.

FIG. 18 depicts an exemplary variation of a method for authenticating user account access within an AI environment.

FIG. 19A is an exemplary variation of a GUI relating to authenticating user account access using an authentication code.

FIG. 19B is an exemplary variation of a GUI relating to user account access through a web-based chat platform.

FIGS. 20A and 20B are schematic illustrations of exemplary GUIs relating to integration of an AI medical assistant within a pre-existing website.

FIG. 21 is an exemplary variation of a GUI relating to predicting and providing medical content in response to a user input.

FIG. 22 is an exemplary variation of a method for providing a customized medical content application module.

FIG. 23 is an exemplary variation of a GUI 2300 providing an administrative interface for maintaining medical content record(s) associated with a particular user group, such as a hospital.

FIGS. 24A-24C are exemplary variations of GUIs relating to an oncology treatment cost calculator module customized for a user group.

FIGS. 25A-25C are exemplary variations of GUIs relating to a pediatric resuscitation module customized for a user group.

FIGS. 26A and 26B are exemplary variations of GUIs relating to a pediatric drug dosing calculator module customized for a user group.

FIG. 27 is an exemplary variation of a GUI relating to a drug image database module customized for a user group.

### DETAILED DESCRIPTION

Non-limiting examples of various aspects and variations of the invention are described herein and illustrated in the accompanying drawings.

Overview

Generally, described herein is an artificial intelligence (AI) environment that manages medical information for users such as a healthcare professional (e.g., physician, nurse, etc.). In some variations, the AI environment may include an electronic medical record platform and an AI medical assistant system. One or more users may interact with a user interface on a user computing device (e.g., mobile device such as a mobile phone or tablet, or other suitable computing device such as a laptop or desktop computer, etc.) that is in communication with the AI environment.

In some variations, a user may engage in chat conversations within the AI environment and/or one or more other users. For example, the AI medical assistant system may be configured to interpret and respond to user input such as user queries for medical information in a readily accessible manner through a machine-implemented conversation simulator such as a chatbot. User input may, for example, request information regarding drugs (e.g., drug description, dosage guidelines, drug interactions, etc.), diseases, medical calculators, etc. As another example, a user may additionally or alternatively communicate with other users over a network through the user interface, such as to share medical information (e.g., over chat conversations, by sharing files such as PDFs, other document files, images or videos, by sharing links to content, etc.) and/or otherwise collaborate on medical care for a patient. At least some of the medical information relating to a patient may be automatically identified by the AI medical assistant system as suitable for storage in an electronic medical record for the patient and subsequently automatically stored in the electronic medical record. For example, one or more predictive algorithms can interpret user input as queries and determine the most relevant results and/or options to display based on the user query and identified relevant content, as further described below.

Additionally or alternatively, the user interface may enable a user to contribute medical information to an electronic medical record for a patient such as through verbal and/or audio-based notetaking, or other designation of medical information for storage in an electronic medical record. Furthermore, as described in detail herein, a user may train the AI medical assistant system medical knowledge based on existing content and/or other content such as user-generated content (e.g., generated through dialogue with a conversation simulator such as that described below, photos taken by one or more users with a user computing device such as a mobile phone, content dictated by one or more users with a microphone, etc.). Such training may, for example, continually improve users' ability to access medical information provided within the AI environment. For example, in some aspects, a user may train or teach the AI medical assistant new medical knowledge or content through a process of manually selecting content, tagging and labeling the content of interest, and instructing the AI medical assistant to index and store this content within a virtual archive. The content may subsequently be easily recalled from the virtual archive by one or more users within the AI platform (e.g., through the AI medical assistant or otherwise).

Furthermore, the AI environment may include a content management platform including a system of web applications that allows entities (e.g., healthcare institutions, organizations, and/or other entities associated with user groups) to easily create, add and/or updated customized medical content in real-time for users to then search within the AI environment, such as with the AI medical assistant system. For example, the content management platform may include one or more content modules with user group-specific content (e.g., call rosters, drug formularies, physician directory information, hospital guidelines and protocols, videos, images, continuing medical education (CME) materials, etc.). The AI medical assistant system may be synchronized with the content management platform, and may be trained through a tagging and indexing process (e.g., by the entities managing the content modules through the content management platform) similar to that described above and described in further detail below.

The AI environment may be accessible in multiple manners. For example, in some variations the AI medical assistant may include a conversation simulator accessible on a mobile chat platform (e.g., accessible through a mobile application executable on a mobile computing device such as a smartphone) as well as a custom web-based platform (e.g., accessible through a web browser on a laptop or desktop computing device). In these variations, a user can interact with the mobile and web-based platforms interchangeably to instantly create, add, and/or search medical content (including entity-specific content, personal content, medical resources, etc.) associated with their user account. As another example, the AI medical assistant may be integrated within pre-existing websites and/or mobile applications, and accessible by selection of an icon (e.g., button) displayed within the website or mobile application user interface, or in any other suitable manner.

Accordingly, in some variations the methods and systems described herein may enable easy and efficient access to medical information (from medical resource databases, medical institutions or other organizations, user-generated content, electronic medical records, other members of a patient care team, etc.), thereby improving medical care and treatment of patients.

### AI Environment

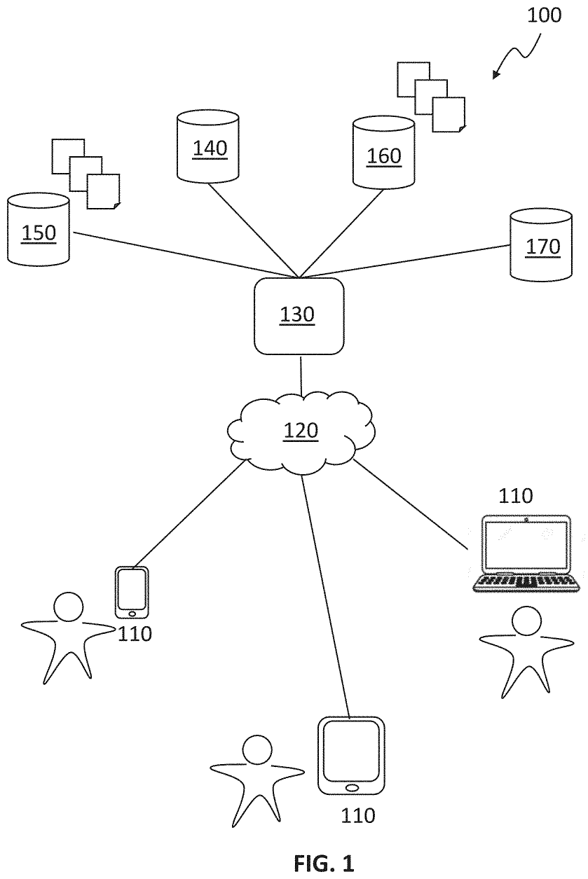

FIG. 1 illustrates an exemplary architecture for an AI environment 100. As shown in FIG. 1, one or more user computing devices 110 are operated by respective users (e.g., physicians, nurse practitioners, nurses, medical assistants, etc.) and may be communicatively connected to a network 120. As described in further detail below, each of the user computing devices may be configured to communicate with other user computing devices 110 within the AI environment 100. An AI medical assistant system 130 may also be communicatively connected to the network 120 to provide medical-related information to one or more users. The medical assistant system 130 may be communicatively connected to one or more medical content sources providing such medical-related information (e.g., over network 120 or the like).

For example, the medical assistant system 130 may be communicatively coupled to one or more medical resource databases 140 that the medical assistant system 130 may access for medical information. As another example, the medical assistant system 130 may be communicatively connected to an electronic medical record (EMR) database 150 configured to store electronic medical records for one or more patients, such that a user computing device 110 and/or the medical assistant system 130 may be configured to read and/or write information to electronic medical records over the network 120. As another example, the medical assistant system 130 may be communicatively connected to one or more clinic modules 160 that may include information specific to a clinic or other medical institution (e.g., drug formulary or pharmacy information, lab medicine, call rosters, physician directory information, hospital guidelines and protocols, videos, images, continuing medical education (CME) quizzes, etc.). As yet another example, the medical assistant system 130 may be communicatively coupled to one or more user libraries which may include user-generated information. As another example, the medical assistant system 130 may be communicatively coupled to one or more third party application programming interfaces (API) which may enable access to other third party databases or other sources of information (e.g. publicly available content sources, medical content publishers). The medical assistant system 130 may additionally or alternatively be communicatively coupled to any suitable sources of medical information.

### Computing Devices

In some variations, a user computing device 110 may include a mobile computing device (e.g., mobile phone, tablet, personal digital assistant, etc.) or other suitable computing device (e.g., laptop computer, desktop computer, other suitable network-enabled device, etc.).

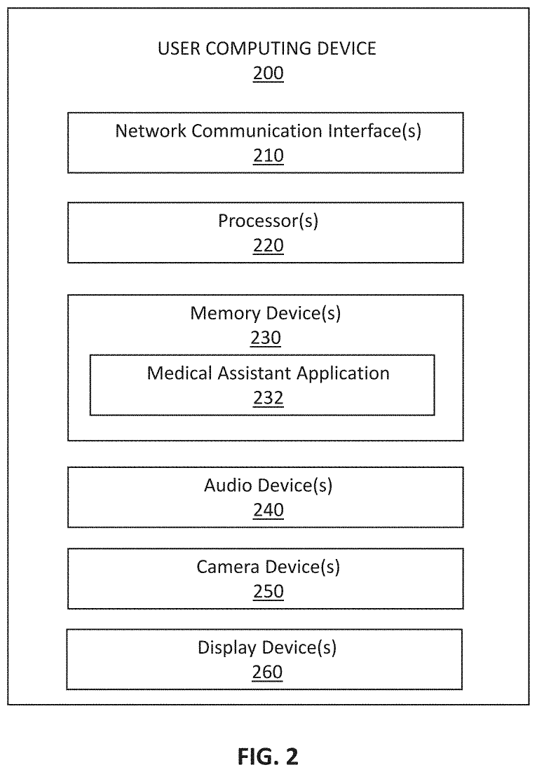

Generally, as shown in FIG. 2 schematically depicting a user computing device 200, the computing devices described herein may include a controller including at least one processor 220 (e.g., CPU) and at least one memory device 230 (which can include one or more computer-readable storage mediums). The processor 220 may incorporate data received from the memory device 230, user input, for example. The memory device 230 may include stored instructions to cause the processor to execute modules, processes, and/or functions associated with the methods described herein. In some variations, the memory device and processor may be implemented on a single chip, while in other variations they can be implanted on separate chips.

The processor 220 may be any suitable processing device configured to run and/or execute a set of instructions or code, and may include one or more data processors, image processors, graphics processing units, physics processing units, digital signal processors, and/or central processing units. The processor may be, for example, a general purpose processor, a Field Programmable Gate Array (FPGA), an Application Specific Integrated Circuit (ASIC), and/or the like. The processor may be configured to run and/or execute application processes and/or other modules, processes and/or functions associated with the system and/or a network associated therewith. The underlying device technologies may be provided in a variety of component types (e.g., MOSFET technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, and/or the like.

In some variations, the memory device 230 may include a database and may be, for example, a random access memory (RAM), a memory buffer, a hard drive, an erasable programmable read-only memory (EPROM), an electrically erasable read-only memory (EEPROM), a read-only memory (ROM), Flash memory, and the like. The memory device may store instructions to cause the processor to execute modules, processes, and/or functions such as measurement data processing, measurement device control, communication, and/or device settings. Some variations described herein relate to a computer storage product with a non-transitory computer-readable medium (also may be referred to as a non-transitory processor-readable medium) having instructions or computer code thereon for performing various computer-implemented operations. The computer-readable medium (or processor-readable medium) may be non-transitory in the sense that it does not include transitory propagating signals per se (e.g., a propagating electromagnetic wave carrying information on a transmission medium such as space or a cable). The media and computer code (also may be referred to as code or algorithm) may be those designed and constructed for the specific purpose or purposes.

Examples of non-transitory computer-readable media include, but are not limited to, magnetic storage media such as hard disks, floppy disks, and magnetic tape; optical storage media such as Compact Disc/Digital Video Discs (CD/DVDs); Compact Disc-Read Only Memories (CDROMs), and holographic devices; magneto-optical storage media such as optical disks; solid state storage devices such as a solid state drive (SSD) and a solid state hybrid drive (SSHD); carrier wave signal processing modules; and hardware devices that are specially configured to store and execute program code, such as Application-Specific Integrated Circuits (ASICs), Programmable Logic Devices (PLDs), Read-Only Memory (ROM), and Random-Access Memory (RAM) devices. Other variations described herein relate to a computer program product, which may include, for example, the instructions and/or computer code disclosed herein.

The systems, devices, and/or methods described herein may be performed by software (executed on hardware), hardware, or a combination thereof. Hardware modules may include, for example, a general-purpose processor (or microprocessor or microcontroller), a field programmable gate array (FPGA), and/or an application specific integrated circuit (ASIC). Software modules (executed on hardware) may be expressed in a variety of software languages (e.g., computer code), including C, C++, Java®, Python, Ruby, Visual Basic®, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. Additional examples of computer code include, but are not limited to, control signals, encrypted code, and compressed code.

In some variations, the memory device 230 may store a medical assistant application 232 configured to enable the computing device 200 operate within the AI environment (e.g., communicate with other computing devices within the AI environment, communicate with a medical assistant system, etc.) as further described herein. The medical assistant application 232 may, for example, be configured to render a text chat interface that facilitates conversation with other users of the medical assistant application 232 on other computing devices, and/or conversation with an AI medical assistant system.

In some variations, a computing device may include at least one communication interface 210 configured to permit a user to control the computing device. The communication interface may include a network interface configured to connect the computing device to another system (e.g., internet, remote server, database) by wired or wireless connection. In some variations, the computing device may be in communication with other devices via one or more wired or wireless networks. In some variations, the communication interface may include a radiofrequency receiver, transmitter, and/or optical (e.g., infrared) receiver and transmitter configured to communicate with one or more device and/or networks.

Wireless communication may use any of a plurality of communication standards, protocols, and technologies, including but not limited to, Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (WiFi) (e.g., IEEE 802.11a, IEEE 802.11b, IEEE 802.11g, IEEE 802.11n, and the like), voice over internet Protocol (VoIP), Wi-MAX, a protocol for e-mail (e.g., internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol.

The communication interface 210 may further include a user interface configured to permit a user (e.g., patient, health care professional, etc.) to control the computing device. The communication interface may permit a user to interact with and/or control a computing device directly and/or remotely. For example, a user interface of the computing device may include at least one input device for a user to input commands and/or at least one output device for a user to receive output (e.g., prompts on a display device). Suitable input devices include, for example, a touchscreen to receive tactile inputs (e.g., on a displayed keyboard or on a displayed UI), and a microphone to receive audio inputs (e.g., spoken word).

Suitable output devices include, for example, an audio device 240, a display device 260, and/or other device for communicating with the patient through visual, auditory, tactile, and/or other senses. In some variations, the display may include, for example, at least one of a light emitting diode (LED), liquid crystal display (LCD), electroluminescent display (ELD), plasma display panel (PDP), thin film transistor (TFT), organic light emitting diodes (OLED), electronica paper/e-ink display, laser display, holographic display, or any suitable kind of display device. In some variations, an audio device may include at least one of a speaker, a piezoelectric audio device, magnetostrictive speaker, and/or digital speaker. Other output devices may include, for example, a vibration motor to provide vibrational feedback to the patient. In some variations, the user computing device 200 may include at least one camera device 250, which may include any suitable optical sensor (e.g., configured to capture still images, capture videos, etc.).

### Network

In some variations, the systems and methods described herein may be in communication via, for example, one or more networks, each of which may be any type of wired network or wireless network. A wireless network may refer to any type of digital network that is not connected by cables of any kind. Examples of wireless communication in a wireless network include, but are not limited to, cellular, radio, satellite, and microwave communication. However, a wireless network may connect to a wired network in order to interface with the Internet, other carrier voice and data networks, business networks, and personal networks. A wired network may be carried over copper twisted pair, coaxial cable and/or fiber optic cables. There are many different types of wired networks including wide area networks (WAN), metropolitan area networks (MAN), local area networks (LAN), internet area networks (IAN), campus area networks (CAN), global area networks (GAN) like the internet, and virtual private networks (VPN). “Network” may refer to any combination of wireless, wired, public, and private data networks that may be interconnected through the internet to provide a unified networking and information access system. Furthermore, cellular communication may encompass technologies such as GSM, PCS, CDMA or GPRS, W-CDMA, EDGE or CDMA2000, LTE, WiMAX, and 5G networking standards. Some wireless network deployments may combine networks from multiple cellular networks or use a mix of cellular, Wi-Fi, and satellite communication.

### Medical Content Sources

The medical assistant system 130 may be communicatively connected to one or more medical content sources to enable a user to create, add, and/or search medical content in real-time or substantially real-time. Furthermore, as described in further detail below, the content in any one or more of the medical content sources may be used to train the AI medical assistant system to improve the ability of the system to determine and provide the most relevant content to users, such as in response to a user query.

For example, as shown in FIG. 1, the one or more medical content sources may include one or more medical resource databases 140, such as medical encyclopedias (which may include content that is publicly available and/or subscription-based), drug databases, guidelines, protocols, other publications, calculators, and the like. Accordingly, the medical assistant system 130 may access the medical content contained in such medical resource databases, so as to provide suitable medical content to a user (e.g., in response to a user query through the conversation simulator).

As another example, the one or more medical content sources may include at least one electronic medical record (EMR) database 150 configured to store electronic medical records for one or more patients, such that a user computing device 110 and/or the medical assistant system 130 may be configured to read and/or write information to electronic medical records over the network 120. Such information may include, for examples, notes or other text, audio (e.g., voice recordings), images, videos, etc. to be associated with a patient's electronic medical records.

As yet another example, the one or more medical content sources may include at least one user libraries which may include user-generated information such as notes or other text, audio (e.g., voice recordings), images, videos, etc. that a user may wish to keep for reference and/or for sharing with other users. User-generated information may be organized into groups and/or subgroups (e.g., albums, folders, etc.).

In some variations, one or more medical content sources may be accessible via a third party API. For example, the one or more medical content sources may include one or more databases or content sources which may be separately managed by a third party, such as billing or claims information, patient scheduling, remote monitoring services (e.g., remote health monitoring services), etc. As an illustrative example, the medical assistant system may be communicatively connected to an API for software systems associated with a health maintenance organization (HMO) to obtain and process billing and claims information. In this example, a doctor or other user may provide the medical assistant system (e.g., via the AI medical assistant system) with input information for Letters of Authorization, and the AI medical assistant system may communicate with the HMO's API to generate and provide and/or store the appropriate Letters of Authorization using the input information (e.g., store in an EMR).

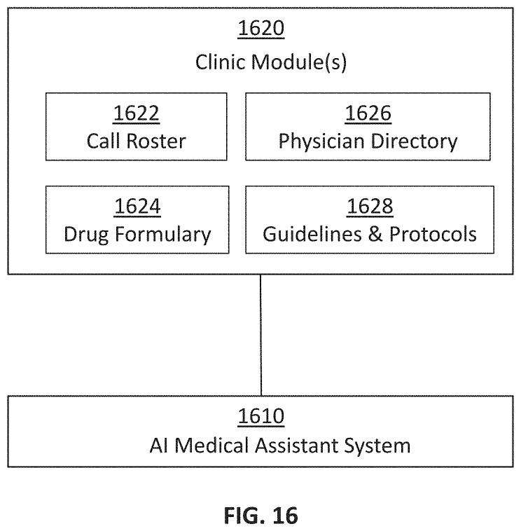

FIG. 16 schematically illustrates another example of a medical content source including one or more clinic modules 1620. Each clinic module 1620 may be associated with a medical institution or other entity with a particular defined user group, such as a hospital, clinic, telemedicine platform, medical association or other organization, etc. Multiple clinic modules 1620 may be maintained on a content management platform as a system of web applications. An AI medical assistant system 1610 may be communicatively coupled to one or more clinic modules 1620. A clinic module 1620 may, for example, include data specific to a clinic or other medical institution, such as a call roster 1622, a drug formulary 1624, a physician directory 1626, hospital guidelines and protocols 1628, videos, images, CME materials, or other suitable information that may be useful for a user to access within the AI environment. Additionally or alternatively, a clinic module 1620 may include patient-facing content such as physician schedules, information on drugs or diseases (e.g., home treatments), educational videos or other informative content, etc. Additionally or alternatively, a clinic module 1620 may include a medical content application module that is modified (e.g., customized) using medical content specific to the medical institution or other entity. For example, a user may access such patient-facing content on his or her device using the AI medical assistant system, for showing or sharing the patient-facing content directly to a patient.

In some variations, a clinic module 1620 associated with a medical institution may be managed through one or more administrative accounts associated with the medical institution. For example, an administrator of the medical institution may use an administrative account to log into the content management platform, such as to create and/or update information in the clinic modules. An administrator may create content modules for their institution based on their specific needs. Each clinic module may be associated with at least one datasheet (e.g., spreadsheet, PDF, or other suitable file type) containing information for that clinic module. As described in further detail below, content of the clinic module (e.g., in the datasheet) may be tagged so as to train the AI medical assistant system with the content as part of the content creation and upload process.

Furthermore, an administrator may update the clinic modules as needed. Updates may include additions, deletions, or other changes to the content in the datasheets. In some variations, updates to the clinic modules may be reflected in real-time (or substantially real-time) in that changes to the clinic modules may immediately affect information that is accessible by the AI medical assistant system 1610 during the course of user operation. For example, in some variations, changes to tags associated with content of the clinic module may immediately affect how the AI medical assistant system characterizes the content. Additionally or alternatively, at least some administrator updates may incur a waiting period before being reflected in the AI environment, such as until a second administrator provides additional approval of the updates, or until a predetermined period of time has passed (e.g., completion of a 24-hour “refresh” cycle or other cycle of suitable duration). For example, based on clinic module settings, certain categories of clinic module updates (e.g., clinical substance of guidelines or protocols) may require approval by a second administrator to ensure accuracy and/or prevent inaccurate tampering with medical content. Depending on clinic module settings, other categories of clinic module updates (e.g., typographical corrections or other minor changes) may not require approval by a second administrator.

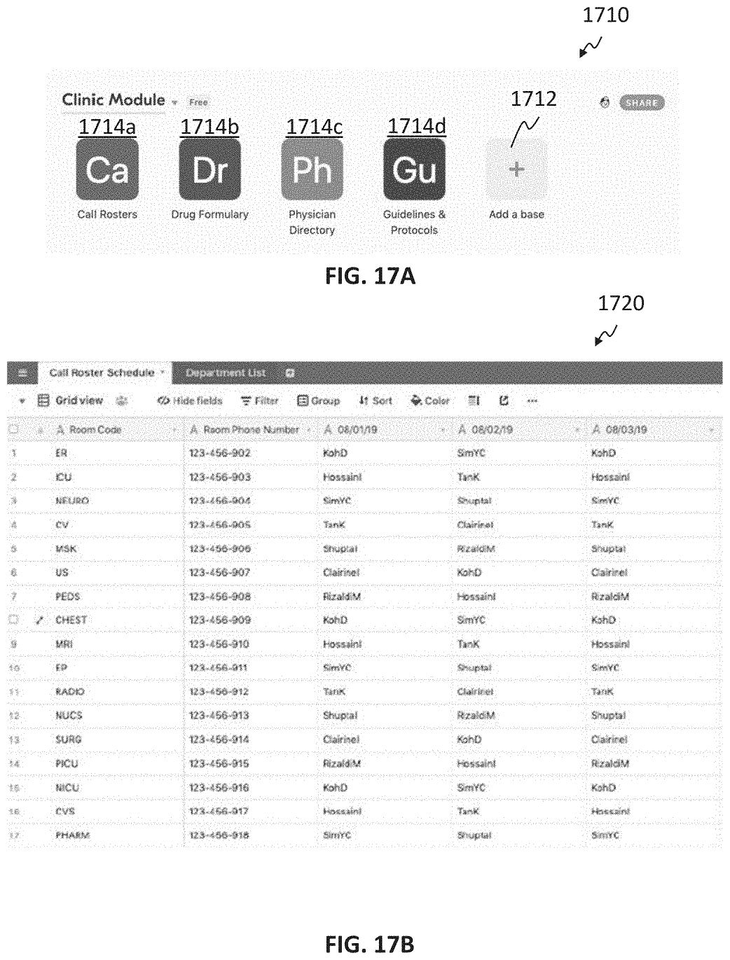

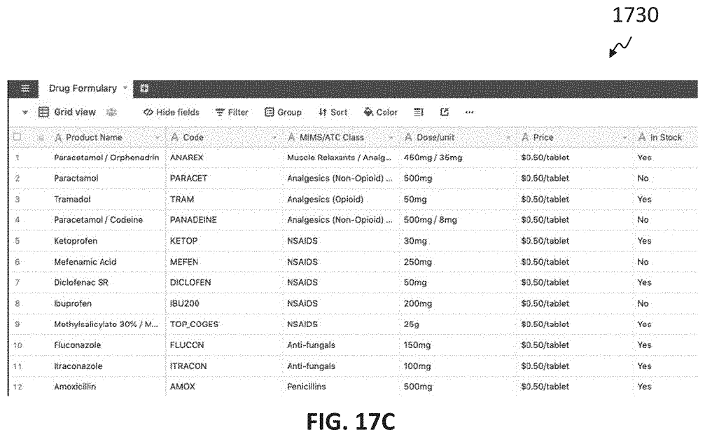

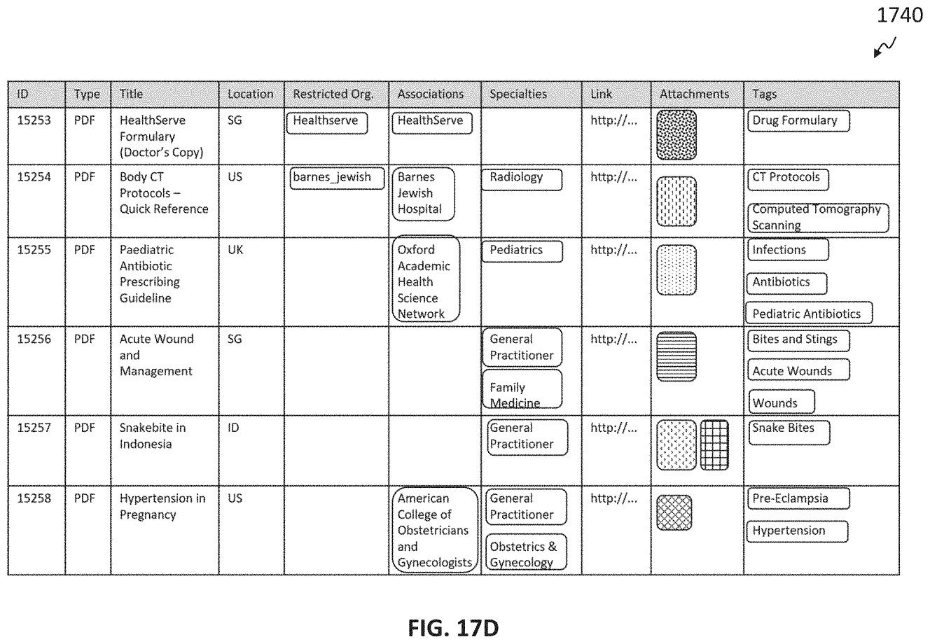

FIG. 17A depicts an exemplary GUI 1720 that may, for example, be displayed to an administrator on a computing device after the administrator successfully logs into the administrative account for managing clinic modules. Icon 1712 may be selected in order to add a new clinic module to the content management platform associated with the administrator's medical institution. For example, after selecting icon 1712, a new datasheet may be uploaded to provide content for a new clinic module. The administrator may furthermore determine clinic module settings, such as provide a name for the clinic module, identify administrative accounts that are permitted to modify or delete the clinic module, etc. Additionally, the administrator may choose whether to make content of the clinic module public, or gated to permit only verified users to search content of the clinic module (and/or define other suitable permission settings).

FIGS. 17B-17D depict exemplary datasheets illustrating exemplary content for clinic modules. FIG. 17B depicts an exemplary datasheet 1720 for a call roster clinic module associated with a hospital. The datasheet 1720 includes information for various rooms or departments in the hospital (e.g., ER, ICU, etc.), contact information (e.g., room phone number) for that room, and the identity of a physician that is on call for that room or department for various days.

FIG. 17C depicts an exemplary datasheet 1730 for a drug formulary clinic module associated with a hospital. Datasheet 1730 includes information for drugs that are approved or otherwise available at the hospital, such as product name, code, dosage, availability in inventory, price, etc. Datasheet 1730 may, for example, be updated in real time for inventory management, and product availability may be instantly searchable through the AI medical assistant system.

As yet another example, FIG. 17D depicts a schematic of an exemplary datasheet 1740 for a guidelines and protocols clinic module associated with a hospital. The datasheet 1730 includes various guidelines and protocols for different clinical needs, and may include multiple fields for each guidelines or protocol to provide context (which may, for example, be used for indexing the content for search and retrieval by the AI system). Exemplary fields include location associated with each item (e.g., country), title, file type, relevant department, tags, etc. Furthermore, the datasheet 1730 may store a copy of file attachments containing the guidelines or protocols, which may be displayed on a computing device (e.g., upon user request). Additionally or alternatively, other suitable kinds of attachments (e.g., links, videos, images, etc.) may be provided as attachments as part of the clinic module.

GUI 1710 shown in FIG. 17A also includes modular icons 1714a-1714d each representing a respective clinic module with medical institution-specific content, and an additional modular icon 1730 permitting creation of a new clinic module. By selecting one of the modular icons representing an existing clinic module, an administrator may edit or otherwise manage content for that clinic module. For example, the administrator may select one of the modular icons 1714a-1714d, which may prompt display of an associated datasheet, and the administrator may directly edit (or replace) the datasheet for the selected clinic module.

### AI Medical Assistant System

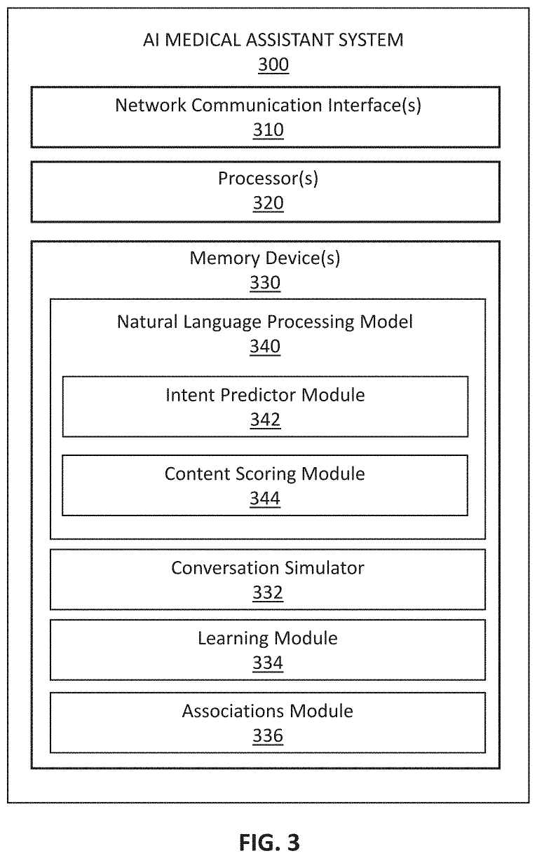

Generally, as shown in the exemplary schematic of FIG. 3, an AI medical assistant system 300 may include at least one network communication interface 310, at least one processor 320, and at least one memory device 330, which may be similar to network communication interface 210, processor 220, and/or memory device 230 described above with respect to FIG. 2. In some variations, one or more servers may host the AI medical assistant system 300 by including the one or more processors 320 and/or one or more memory devices 330.

As shown in FIG. 3, the one or more memory devices 330 may store a natural language processing model 330 (natural language processor) and a conversation simulator 332. The natural language processing model 330 and/or the conversation simulator 332 may be stored on one or multiple memory devices, in any suitable architecture (e.g., distributed, local, etc.). Generally, as further described below, the natural language processing model 330 may be configured to parse user input (e.g., queries or other statements), predict a user intent according to an intent predictor module 342, and attempt to determine suitable medical content associated with the predicted user intent according to a content scoring module 344. The conversation simulator 332 may be configured to emulate human conversation with a user to, for example, communicate information such as medical content in response to user input, or prompt the user for additional information, as further described below. In some variations, the memory device(s) 330 may further include a learning module 334 configured to update and modify the natural language processing model 330 based on supplemental information such as user feedback that characterizes the quality of the medical content provided to the user. Additionally or alternatively, the memory device(s) 330 may include an associations module 336 configured to associate content (e.g., medical content) with one or more tags or other suitable identifiers, such that the associations module may predict queried medical content based on user input (e.g., a request or other input received through the conversation simulator) and provide the predicted medical content to the user. The associations module 336 may, for example include one or more machine learning associations models to be modified, as further described below, by the learning module 334 and/or a suitable machine learning process.

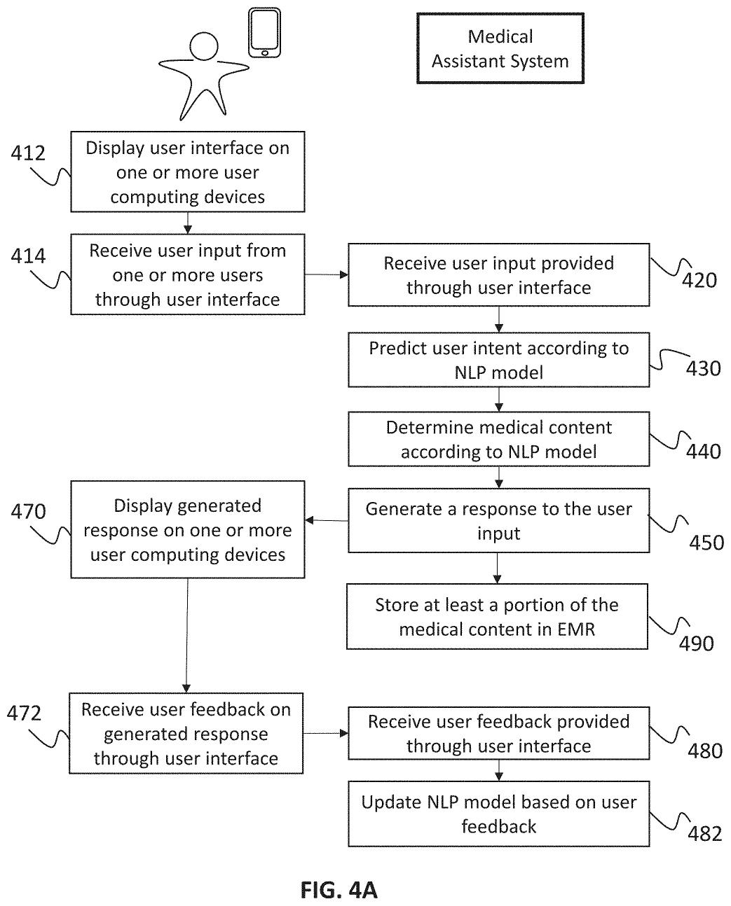

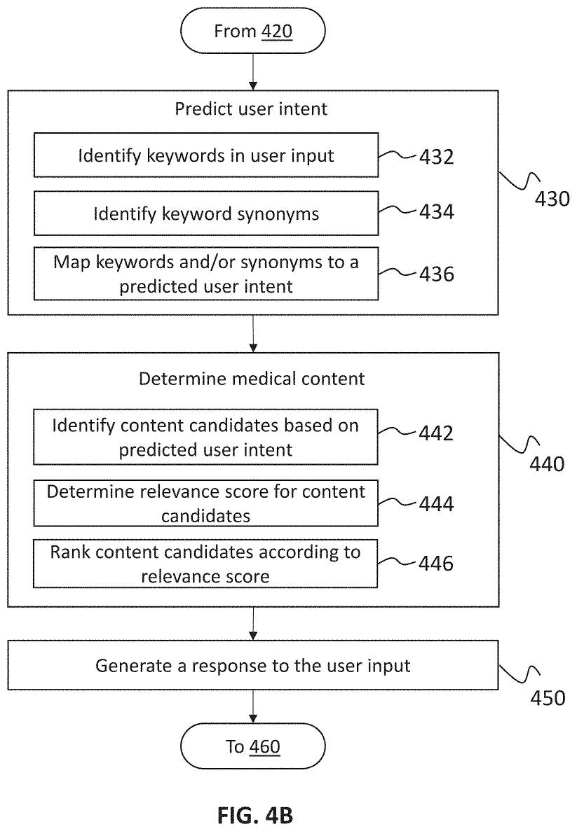

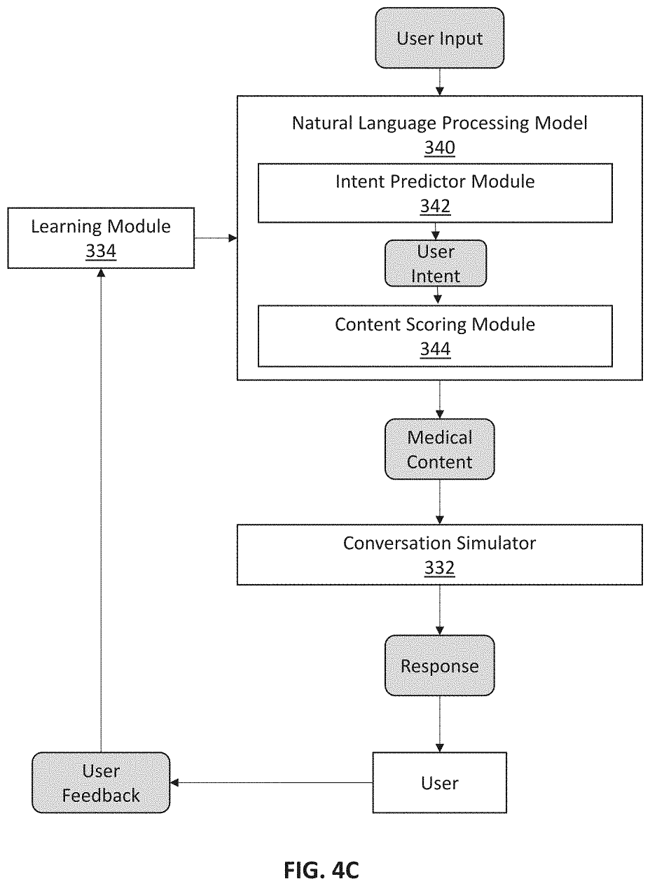

An exemplary interaction between the medical assistant system and a user computing device associated with a user is shown in FIG. 4A. Although the steps and processes of FIG. 4A are ordered in an exemplary sequence, it should be understood that they may alternatively be performed in any suitable order and/or some processes may be performed concurrently.

A medical assistant system (for example, AI medical assistant system 300 described above) may connect to a user interface with a conversation simulator. For example, as shown in FIG. 4A, through a medical assistant application (e.g., loaded on a mobile phone, tablet, etc.) and/or a website interface, a user interface with a conversation simulator may be rendered and displayed on a user computing device (412). For example, the user interface may include a chat interface that may enable text conversations with one or more other users, and/or with the AI medical assistant system. As another example, an interface enabling input of user-entered notes may be displayed on the user computing device. Additional examples of user interfaces are described in further detail below and in U.S. patent application Ser. No. 16/016,330 entitled “METHODS AND SYSTEMS FOR PROVIDING AND ORGANIZING MEDICAL INFORMATION”, which is hereby incorporated in its entirety by this reference.

User Intent and Medical Content Determination

User input may be received through the user interface on the user computing device (414) and provided to the medical assistant system. The medical assistant system may receive the user input (420), such as text- or voice-based input. An intent predictor module (e.g., intent predictor module 342) may process the user input to predict user intent (430), and a content scoring module (e.g., content scoring module 344) may determine medical content (440) from the user intent.

FIG. 4B illustrates one exemplary variation of predicting user intent and determining medical content. As shown in FIG. 4B, after receiving user input provided through the user interface (420), the medical assistant system may identify at least one keyword in the user input (432). Keywords may, for example, be identified based on comparing words against a database of known or predetermined words of importance (e.g., “diagnose”, “calculate”, “treat”, medication or drug names, etc.). In some variations, the medical assistant system may be configured to identify one or more synonyms of identified keywords (434), such as by searching a thesaurus or other suitable database that matches or associates keywords with related meanings. The synonyms may, in some variations, be used to expand the range of variety of medical content candidates that may be mapped to the user intent.

The medical assistant system may be configured to map at least a portion of the keywords and/or synonyms of keywords to a predicted user intent (436). For example, the intent predictor module may include or be associated with a natural language processing (NLP) model that is trained to associate a word to a predicted user intent. The NLP model may, for example, incorporate a suitable machine learning model or suitable NLP that is trained on a training dataset including vetted or identified associations between keywords and meanings, and/or user feedback that updates or improves associations between keywords and meanings (e.g., as described in further detail below). Furthermore, the NLP model may additionally or alternatively be trained at least in part on a stored dictionary and/or thesaurus, which may include, for example, synonyms including alternative terminology and/or other aspects of language derived from user interaction (e.g., user queries) with the medical assistant system. Accordingly, the NLP model may be configured to map words such as a keyword in the user input (and/or a synonym of the keyword) to at least one predicted user intent.

Potential medical content can be identified based at least in part on the predicted user intent (442). Medical content may be identified by matching the predicted user intent to various content in a medical content sources (e.g., user's library, clinic modules, publicly available content, etc.). For each content candidate, a relevance score or other metric may be determined (444) such as by a content scoring module 344, where the relevance score characterizes the relevance of that content to the predicted user intent. The relevance score may be expressed numerically and on any suitable scale (e.g., 0-100, 0-50, 0-10, etc.), or in any suitable manner.

Generally, as described in further detail below, content candidates may be ranked using one or more search relevance algorithms, which may be based on relevance scores depending on a combination of one or more various factors. For example, a relevance score for a content candidate may be at least partially based on overlap or similarity between the user's search query and the content's metadata (e.g., title, tags, authors, description, etc.).

As another example, a relevance score for a content candidate may be at least partially based on overlap or similarity between the user's search query and chapter or section titles within a document. Chapter and section titles may be automatically identified in a document based on, for example, formatting (e.g., increased boldness, left-justified text, consecutive capitalization, etc.) and/or content characteristic of a title (e.g., numeral or letter followed by text, a segment of text below a predetermined threshold length, etc.). Certain chapters or sections may furthermore be ranked in importance when determining the relevance score for a content candidate. For example, an abstract or introduction section of a document may be weighed more heavily than a “references cited” section of the document. Accordingly, in some variations, ranking of relevance may be performed at a chapter or section level of a document instead of at a higher document level, such that the selection of content for return to the user is based on chapters or sections of a document, rather than individual documents. Furthermore, in some variations, a relevance score for a content candidate in the form of a video may be at least partially based on bookmarked or labeled scenes in the video (rather than overall title of the video).

In some variations, content candidates may additionally or alternatively be ranked in view of a stored dictionary/thesaurus that may include synonyms and/or other word associations that may be continually improved through suitable algorithms through user interaction and feedback. For example, the stored dictionary/thesaurus may be trained at least in part on previous user queries, user feedback (e.g., user approval rating of interpretation of their query and/or presented content mapped to their query), and/or other user interactions (e.g., which presented content the user actually selects). In some variations, the search relevance algorithms may additionally or alternatively take into account different media types (e.g., videos, images, guidelines, textbooks, etc.) such as if a certain media type appears in the user query.

Additionally or alternatively, in some variations, the relevance score for a content candidate may be based on word similarity between the content and the user intent (e.g., similarity in meaning, semantics, and orthography such as spelling, etc.). Different words may have different weighting factors to scale the significance of a word when assessing word similarity between content and user intent. Another factor affecting relevance score for a content candidate may be syntax structure (e.g., sentence structure). For example, a user input of “patient experienced pain in the abdomen” has a syntax structure that suggests pain in the abdomen rather than patient in the abdomen. Accordingly, diagnostic and/or treatment content relating to pain in the abdomen may have a higher relevance score than other kinds of medical content. As another example, a user input of “64 slice GE lightspeed abdomen pelvis CT protocols” has a syntax structure that is less likely to suggest 64 things, but more likely to suggest a specific machine protocol for a particular machine brand and technology (GE LIGHTSPEED computed tomography) with a specific number of slices (64) and a specific anatomical region (abdomen, pelvis). Accordingly, protocol content for these parameters may have a higher relevance score than other kinds of medical content.

Other suitable factors affecting relevance score for a content candidate may include suitable rules or algorithms based on user studies, user feedback, etc. For example, content candidates including known acronyms of user intent may have lower relevance scores. As another example, colloquial or shorthand medical terminology may be “learned” by user feedback and used to adjust relevance scores appropriately. In some variations, the content scoring module 344 may include the NLP model in communication with or accessing one or more suitable medical resource databases, and the NLP model may be configured to identify content candidates and/or determine relevance scores for content candidates. Furthermore, the relevance scores for multiple content candidates may be ranked (446) (e.g., sorted according to relevance score) in order to identify medical content most likely to be associated with the predicted user intent.

In some variations, user intent and/or medical content may additionally or alternatively be predicted based at least in part on a user's previous search history and/or previous terminology (in chat conversations, notes taken by the user, files in their user library, description or tags of images and/or videos taken by the user, etc.). For example, for a particular user, the system may be more likely to predict user intent and/or identify medical content that is similar to the user's previous search history and/or terminology. As an illustrative example, when predicting the intent of a user input from a user who frequently searches for drug information, the intent predictor module may be more likely to predict a user intent that is drug-related. Additionally or alternatively, when determining medical content for such a user, the content scoring module may generate relevance scores that are higher for content that is drug-related. Similarly, a user's typical terminology (e.g., typically referring to a drug as “acetaminophen” instead of paracetamol or by a brand name therefor) may inform the prediction of user intent and/or determination of medical content for that user. Thus, incorporation of such user-specific data may be useful, for example, to help distinguish between multiple options for intent and/or content that otherwise are similarly likely to be appropriate (e.g., user-specific data may be a “tie-breaker” to help choose between multiple or ambiguous options).

Additionally or alternatively, user intent and/or medical content may be predicted or determined based at least in part on one or more user characteristics, such as geolocation or nationality. Accordingly, geographically-relevant data may help inform the intent predictor module and/or the content scoring module. For example, users located in (or originating from) different geographical locations or hospital institutions (or other medical institution or user group) may refer to the same drug in different ways or have clinical practice guidelines specific to their location or hospital (or other medical institution). Accordingly, a user's location and/or nationality (e.g., drawn from a GPS-enabled user computing device, IP address of the user computing device, and/or user profile, etc.) and/or the user's medical institution or other user group with which the user is associated, may inform the prediction of user intent and/or determination of medical content for that user. As another example, users located in (or originating from) different geographical locations may use medical terminology that is characteristic of local medical association guidelines. Thus, incorporation of geographically-relevant data may be useful to help distinguish between multiple options for intent and/or content that otherwise are similarly likely to be appropriate (e.g., user-specific data may be a “tie-breaker” to help choose between multiple or ambiguous options). As another example, medical content candidate(s) associated with a user group of the user (e.g., derived from, originating from, or otherwise associated with the user group) such as the user's hospital institution, may be scored with a higher relevance score than, for example, generic information.

As shown in FIGS. 4A and 4B, a response to the user input may be generated (450) based at least in part on the ranked relevance scores for content candidates. For example, a content candidate with the highest relevance score may be considered the most suitable content associated with the predicted user intent, and provided in a response to the user. The most relevant content may, for example, be displayed on the user interface of the user computing device (470). The most relevant medical content may be quoted directly from the medical resource database along with a citation, and presented to the user in the user interface on the user computing device. Additionally or alternatively, in some variations, a conversation simulator 332 may be configured to receive the medical content associated with the predicted user intent (e.g., from the content scoring module 344) and generate a suitable response to the user input. For example, the conversation simulator 332 may be configured to present the medical content in a colloquial manner. Furthermore, in some variations, the generated response may include an invitation or opportunity for the user to “click through” to obtain additional related medical content. For example, the generated response may, in some instances, include only a selected portion (e.g., first paragraph, summary, etc.) of the medical content. The displayed response may be accompanied by a hyperlink that, when selected, may allow the user to access additional portions of the medical content (e.g., the displayed generated response may enable a user to “click through” to view the rest of the medical content beyond the quoted content).

In some variations, the content candidate with the highest relevance score may be selected as the most suitable content to provide to the user only if a confidence score is sufficiently above a predetermined threshold. A confidence score may be based on, for example, a statistical characteristic of the distribution of relevance scores among the content candidates (e.g., characterizing the highest relevance score as being sufficiently greater than the second-highest relevance score).

In some instances, a generated response to the user input may include multiple content candidates. For example, if two or more content candidates have relevance scores that are greater than a predetermined threshold (and/or there is insufficient confidence that any single one of the content candidates is the “best” content for responding to the user input), then multiple content candidates may be provided to the user. Upon display of the generated response with multiple content candidates (470), the user may be presented with the option to select one of the content candidates for proceeding. In some variations, a conversation simulator 332 may be configured to prompt the user to select among multiple content candidates.

Furthermore, in some instances, a generated response to the user input may include a follow-up query to the user to obtain additional information. For example, the follow-up query may seek to clarify user intent within the context of potential content candidates. In one illustration, the medical assistant system may identify a user intent of obtaining dosage information for a particular medication. In response, the system may generate a follow-up query to the user to clarify whether the user seeks dosage information for an adult patient or a pediatric patient. Upon receiving additional user input in response to the follow-up query, the medical assistant system may similarly parse and process the input to identify suitable medical content as described above.

In some instances, such as if no suitable content candidate is determined (e.g., no content candidate has a sufficiently high relevance score), then a generated response to the user input may omit suitable content. Instead, the generated response may include an indication that analysis of the user input was inconclusive (e.g., display a phrase such as “I don't know” or “Please rephrase your question”).

As shown in FIG. 4A, in some variations, the medical assistant system may be configured to store at least a portion of medical content (490) in an electronic medical record associated with the patient. For example, if a user queries to the medical assistant system about suitable medication dosage for a patient, the medical assistant system may generate a response to the user query (450) including content relating to the suitable medication dosage, and then record in the patient's electronic medical record the appropriate medication dosage. As another example, the medical assistant system may receive and process information from an electronic medical record (e.g., receive current prescription information for a patient, and apply a drug interaction checker to the current prescription information and/or prospective new prescription information). Additionally or alternatively, the medical assistant system may provide a result associated with the processed information (e.g., display the results of the drug interaction checker to one or more users) and/or store a result associated with the processed information to the electronic medical record associated with the patient.

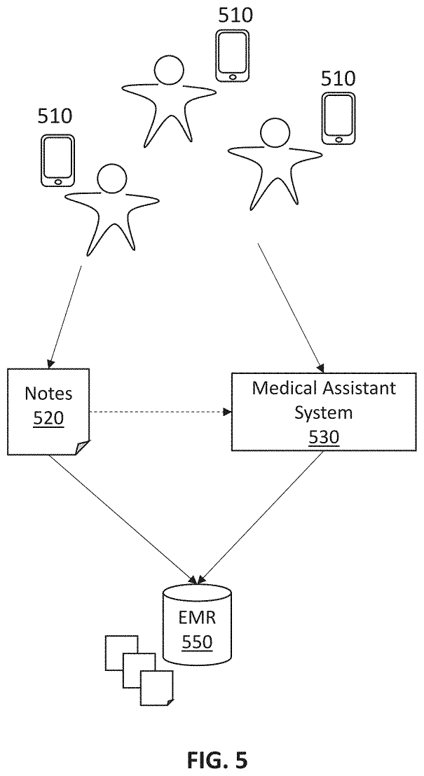

Although the operation of an AI medical assistant system is described herein primarily in the context of the AI medical assistant corresponding with a single user, it should be understood that interpretation of user input and generation of suitable responses to the user input may be applied in other contexts. For example, as further described below, user input may be in the form of dialogue or group chats between different users. Furthermore, as shown in the schematic of FIG. 5, the AI medical assistant system 530 may be configured to receive user input of various kinds (e.g., within simulated conversation between the AI medical assistant and a single user 510, simulated conversation between the AI medical assistant and multiple users, conversation between multiple users on user computing devices 510 within the medical assistant application, etc.). The AI medical assistant system 530 may interpret the user input and store generated medical content in an electronic medical record 550 associated with the patient, similar to that described above.

As another example, as shown in FIG. 5, the AI medical assistant system may be configured to receive user input in the form of a note 520 (e.g., freestyle note or pre-organized case note that may be typed and/or spoken by a user) and identify suitable content for storage in an electronic medical record 550 associated with a patient. Notes may additionally or alternatively be directly stored or associated with the electronic medical record.

### Training the AI Medical Assistant System

In some variations, the AI medical assistant system may be modified over time through one or more suitable training processes. For example, training the AI medical assistant system may help improve accuracy of the system when interpreting a user input (e.g., query) and/or determining medical content in response to the user input.

### User Training

In some variations, user input can be used to train one or more aspects of the AI system. For example, user input (e.g., feedback) may be used to train the intent predictor module, such as by training the NLP model. As shown in FIG. 4A, user feedback on the generated response may be received through the user interface on the user computing device (472). The feedback may include, for example, a numerical or graphical rating of the usefulness or accuracy of the generated response (e.g., rating of 1-5, rating of a discrete number of stars, thumbs up, thumbs down, upvote, downvote, etc.). As another example, the feedback may include a text-based comment (e.g., “Helpful”, “Not helpful”, “Accurate”, “Inaccurate”). Feedback may be received following display of a prompt for such feedback (e.g., “Was this helpful?”). The medical assistant system may receive such user feedback (480) and then update the NLP model or other algorithm based on the user feedback (482).

The feedback process is further illustrated in the schematic of FIG. 4C. As shown in FIG. 4C, an NLP model 340 may receive a user input and analyze it according to an intent predictor module 342 to predict a user intent, which may then be provided to a content scoring module 344 to determine suitable medical content. A conversation simulator 332 may incorporate the medical content and display or otherwise provide the medical content in a response to the user. A user may provide user feedback on the suitability of the generated response, and the user feedback may be provided to a learning module 334 that updates the NLP model and its module components. Accordingly, if the medical assistant system makes a mistake, the user can provide feedback to allow the AI system to learn from its mistakes. As such, user feedback may be used to continuously train and update the AI system. For example, user feedback may be used to adjust weighting factors for words when determining suitable medical content, and/or adjust other factors in the algorithm for determining relevance score for a content candidate. In some variations, the NLP model may be updated for use by all users, such that all users benefit from a modified model that is updated in view of feedback from individual users. In some variations, the NLP model may be updated for use by only a subset of users (e.g., only users in the same practice area as the user providing feedback).

In some variations, some types of user feedback may be weighted or considered more heavily than other types of user feedback. For example, feedback from a more experienced user (e.g., senior physician) may be treated as more influential in updating the NLP model than a less experienced user (e.g., junior physician). As another example, feedback from a user of a particular practice type regarding medical content for that practice type may be treated as more influential in updating the NLP model (e.g., feedback from a radiologist on relevance of a response to a user query regarding diagnostics using medical images may be treated as more influential).

As another example of user input to train the AI system, user input may be used to train a machine learning model providing the AI system with new medical knowledge or other content. Generally, in some variations, one or more users may train or teach an aspect of the AI system (e.g., the AI medical assistant) new content through a process of manually selecting content, tagging or labeling the content of interest, and instructing the AI system to modify a machine learning associations model. The associations model may be configured and continually modified, for example, to learn associations between different content and tags provided by the user through the user interface. Other associations, such as between tags (e.g., to define similarity or other relationships between tags) and between content (e.g., to define similarity or other relationships between different content). The associations model may be used to predict queried content based on user input (e.g., user request, user behavior or interactions with the user interface, etc.), such that the predicted queried content may be displayed or otherwise communicated to the user.

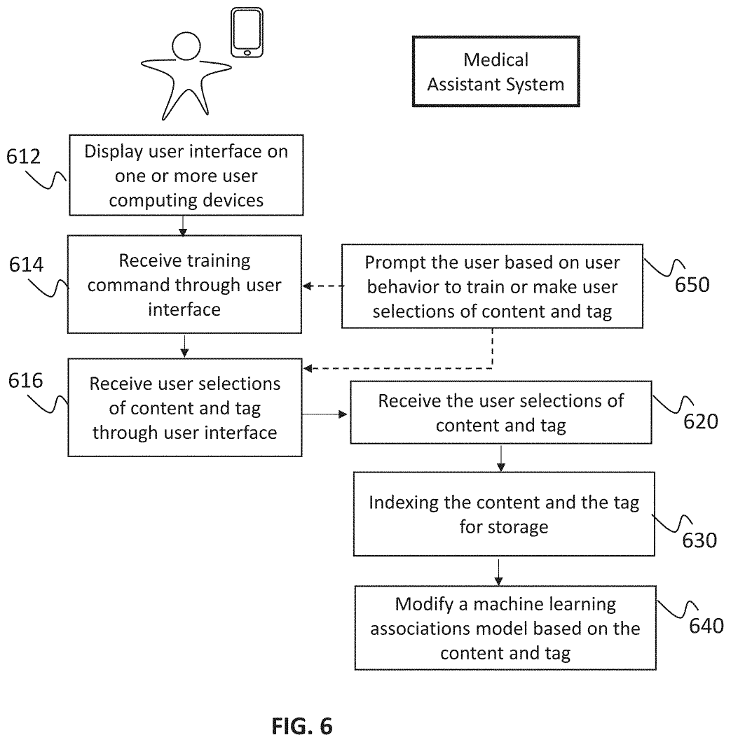

An example of user input applied to train the machine learning associations model is shown in FIG. 6. Although the steps and processes of FIG. 6 are ordered in an exemplary sequence, it should be understood that they may alternatively be performed in any suitable order and/or some processes may be performed concurrently.

A medical assistant system (for example, AI medical assistant system) may connect to a user interface. Similar to that described above with respect to FIG. 4A, the user interface may include a conversation simulator. For example, as shown in FIG. 6, through a medical assistant application (e.g., loaded on a mobile phone, tablet, etc.) and/or a website interface, a user interface with a conversation simulator may be rendered and displayed on a user computing device (612). Various content, such as text, images, videos, files, and/or other media may be obtained from websites, document files, chat message, photo or video archives, and/or other digital sources and be displayed on the user computer device through the user interface.

Generally, user input may be received through the user interface on the user computing device and provided to the medical assistant system for training the associations model. For example, user input may include a training command that is received through the user interface (614), where the training command may be a trigger or a condition for a process to train the associations model. In some variations, the training command may include the selection of an icon, button, or other selectable icon displayed on the user interface. The selectable icon may be, for example, an icon indicative of approval or disapproval (e.g., “thumbs up” or “thumbs down”), a numerical rating, or the like. The selectable icon(s) may be displayed as a response button or bubble within the conversation simulator interface. As another example, the training command may include selection of content for a predetermined amount of time (e.g., selecting and “holding down” the content for a predetermination duration). Additionally or alternatively, the training command may include a text-based or audio-based command (e.g., typed or spoken into a conversation simulator environment), and/or an action-based command (e.g., double-clicking the content, dynamic gesture on the user interface such as tracing a particular shape on the screen of the user computing device, shaking or otherwise moving the user computing device in a predetermined manner, etc.).

In some variations, the user input related to training the associations model may additionally or alternatively include a user selection of content and a user selection of at least one tag to be associated with the selected content (616). The user may select such content for storage and/or future display. The selected content may include, for example, text, images, videos, files, and/or other media. For example, selected content (e.g., medical content) may include medical knowledge (e.g., diagnosis, treatment planning, medication information, etc.), patient information (e.g., medical records, medical images, patient interviews, etc.), or other suitable information that a user may wish to access in the future.

The tag to be associated with the selected content may be an identifier or pointer that helps facilitate access to the selected content. The tag may be, for example, a word, phrase, symbol, and/or other suitable text-based label. The tag may be accompanied with a tag identifier (e.g., “#”, “+”, “*”, user initials, etc.). In some variations, the tag may be another suitable identifier, such as recorded audio (e.g., spoken word or phrase, sound effect, etc.) and/or a gesture on the user interface. A single selected content item may be accompanied by a single tag to be associated with the selected content (1:1 content-tag relationship), or may be accompanied by multiple tags to be associated with the selected content (1:N content-tag relationship). Furthermore, multiple different selected content items may be associated with the same tag.

The training command (614) may be received before or after receiving user selections of content and one or more tags. Additionally or alternatively, in some variations, user behavior (e.g., in the user interface) may be monitored, and certain user behavior may automatically prompt the user to train the associations model (650), such as to input a training command and/or to input a user selection of content and tag(s). The prompt may be issued to the user when the AI medical assistant system determines certain content may be a good candidate to be tagged for easy retrieval. For example, such a prompt to train may be triggered by length of a communicated chat message exceeding a predetermined threshold, as a long chat message may suggest communication of important information (e.g., for a patient medical record). As another example, such a prompt to train may be triggered by the user accessing (e.g., viewing, listening to, etc.) a certain content item a predetermined number of times (or a predetermined frequency) and/or for a predetermined duration, which may indicate usefulness and/or importance of the content item. As yet another example, such a prompt to train may be triggered by a user action such as taking a screenshot, highlighting or otherwise selecting text (e.g., of content in a document viewer, in an internet web browser, etc.), or marking up other displayed content (e.g., circling part of an image). In some variations, the AI medical assistant system may prompt a user to train based on the user's own training history. For example, if the user has previously tagged as “# X-ray” one or more grayscale images that the user has viewed, then the AI medical assistant system may prompt or automatically suggest the user to train the association model to associate a currently-viewed grayscale image with the “# X-ray” tag.

In some variations, the prompt to train, the training command, and/or options for entering one or more tags may be combined in the same dialog box or other user interface element. For example, a single prompt to the user that inquires whether the user would like to tag content can simultaneously display one or more prepopulated, selectable tags and/or a field for entering one or more user-created tags.

Once the user selections of content and tag(s) have been received (620), the user selections may be stored and/or indexed (630) in such a way allowing for efficient retrieval from one or more memory storage devices. The indexing of the content and tags may be performed with any suitable search engine indexing algorithm, such as Elasticsearch. The associations between content and tags, which governs which content or tags are retrieved in response to a user query, may be learned and/or continually modified under an association model (640), such as a machine learning model.

In some variations, the associations model may be modified with new user selections of content and tags. For example, based on the user selections of content and associated tags, the associations model may learn direct relationships between content items and their respective one or more in the form of lookup tables, indexing, etc.

Additionally or alternatively, the associations model may learn, through any suitable machine learning algorithm, associations between different tags, as well as between different content.

Tag-tag associations (i.e., between different tags) may be used to automatically generate and suggest tags to a user, and/or return related content associated with similar tags. For example, user input of one tag may prompt one or more additional tags to be suggested (e.g., displayed) to the user, where the additional tags are generated or identified based on the associations model. The tag-tag associations may also help capture content associated with a tag having a typographical error. For example, if a first content item is tagged with “# surgery” and a second content item is tagged with “# srgery” with the tag inadvertently misspelled (or tagged with other misspellings), a subsequent retrieval by the associations model based on a searched tag “# surgery” may return both the first and second content items, once the association between the tags “# surgery” and “# srgery” is learned by the associations model. Furthermore, one or more tags may be prepopulated as suggested tags based on the content and/or user history (e.g., previously selected tags for similar content).

In some variations, an association between a first tag and a second tag may be learned generally based on degree of similarity between the first and second tags. For example, degree of similarity between tags may be established by identifying a tag (e.g., word, phrase, or symbol following a tag identifier such as “#”) and comparing the tag against a database of synonyms (e.g., such as by searching a thesaurus) and/or comparing the tag against a database of thematic similarity (e.g., a database in which all surgery-based words are associated together). Additionally or alternatively, an association between a first tag and a second tag may be learned generally based on frequency of simultaneous use (or co-occurrences) of the first and second tag for the same content item. For example, the associations model may associate the tags “# X-ray” and “# image” with each other if “# X-ray” and “# image” are selected for the same content (or type of content) for at least a predetermined number of times.

Content-content associations (i.e., between different content) may further inform the automatic generation and suggestion of tags to a user. For example, after a user inputs a tag to be associated with a first content item, the same tag may be suggested by the AI system when the user is preparing to tag a second content item that is associated with the first content item. For example, if a user tags at least one grayscale image with the tag “# X-ray”, then the same “# X-ray” tag may be suggested by the AI system when the user is preparing to tag another grayscale image, once the association among grayscale images is learned by the associations model.

In some variations, an association between a first content item and a second content item may be learned generally based on degree of similarity between the first and second content items. For example, content items of the same content type (e.g., file types such as .jpg, .txt, .pdf) may be associated with each other. As another example, content items of similar subject matter or other features may be associated with each other. Similar subject matter may, for example, include similar image features (arbitrary vectors that encode image properties, such as pixel intensities, red-green-blue (RGB) channel values, contours or lines, etc.), similar content titles (e.g., similar optically-recognized keywords in titles of papers), etc. Images having certain similar image features in common may be associated with one another. For example, associations between different images as depicting blood may be learned if pixel intensities among the different images are red-biased.

### Content Administrator Training

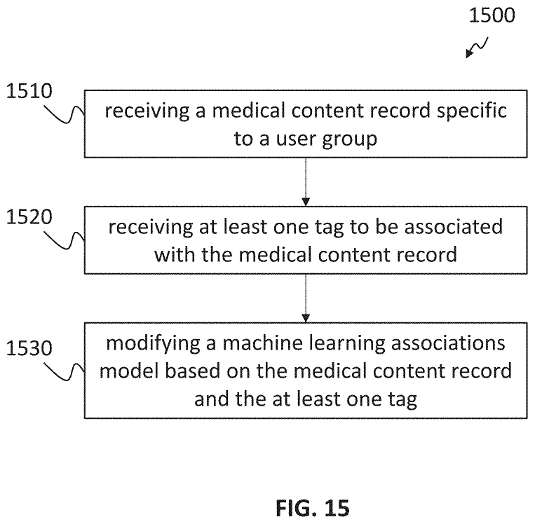

In some variations, input from an administrator of medical content may be used to train the machine learning associations model to provide the AI system with new medical knowledge or other content. Generally, as shown in FIG. 15, in some variations, a method for training the AI medical assistant system may include receiving a medical content record specific to a user group 1510, receiving at least one tag to be associated with the medical content record 1520, and modifying a machine learning associations model based on the medical content record and the least one tag 1530. The associations model may then learn relationships among content such as through tag-tag associations, tag-content associations, and/or content-content associations similar to that described above with respect to training based on user input.

For example, content in clinic modules may be associated with one or more tags as part of the content creation and upload process (and/or with updates to clinic module content by modifying associated datasheets). When clinic module content is tagged, the AI system may also auto-suggest tags using a stored dictionary (e.g., based on known tag-tag associations, tag-content associations, and/or content-content associations, canonical medical terms, curated synonyms, etc.), and an administrator may select one or more of the auto-suggested tags for further labeling of medical content in the clinic module. An administrator may additionally or alternatively choose to add synonyms or new tags to the datasheets or portion thereof, which may further update the stored dictionary with additional synonyms. Furthermore, it should be understood that as various users interact with content of clinic modules, such as by adding user-provided tags, the AI system may subsequently auto-suggest such user-provided tags to an administrator for further tagging of the content. Accordingly, the associations model within the AI environment may continuously evolve through administrative management of clinic modules and/or user engagement with content of clinic modules.

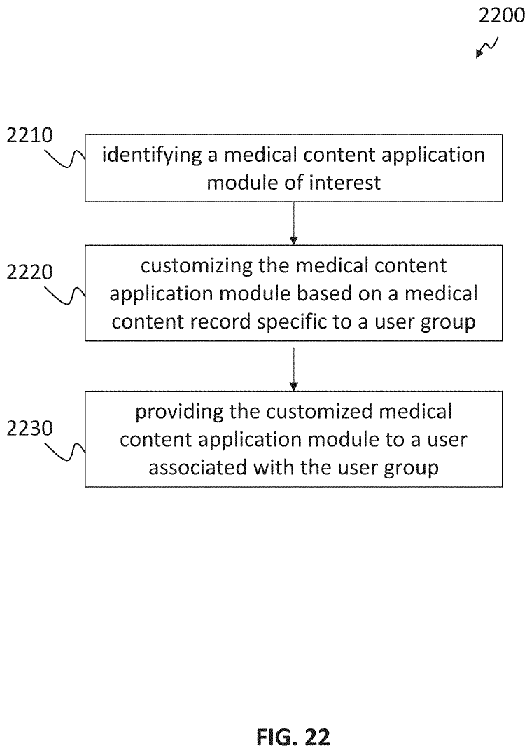

As another example, a clinic module may include a medical content application module that may be customized with a medical content record that is specific to a user group. As shown in FIG. 22, in some variations, a method 2220 may include identifying a medical content application module of interest 2210, customizing the medical content application module based on a medical content record specific to a user group 2220, and providing the customized medical content application module to a user group associated with the user group 2240.

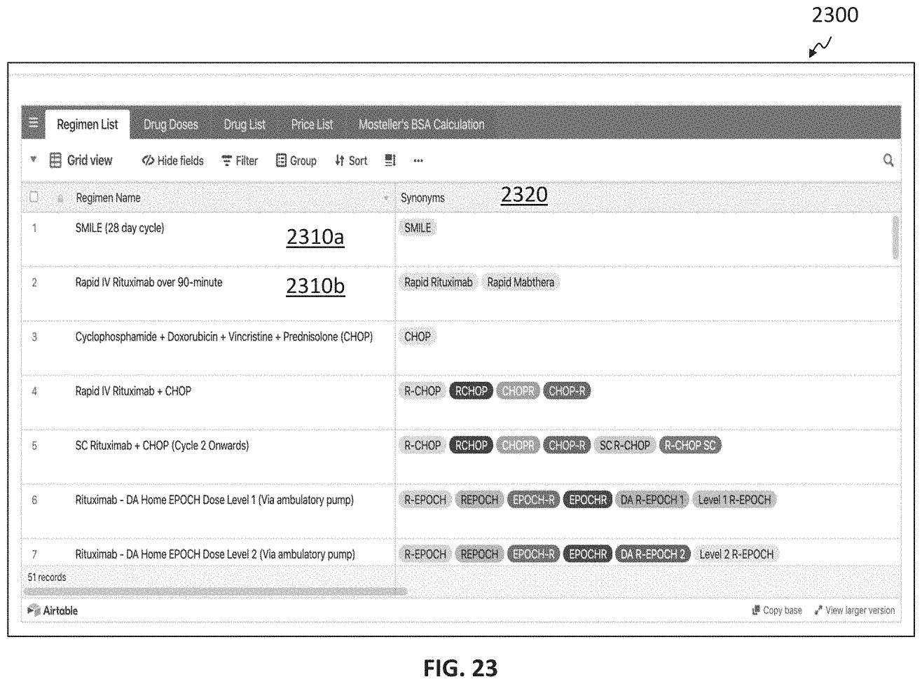

The medical content application module may, for example, enable the AI system to provide medical information that is particularized for users associated with the user group (e.g., medical calculators, hospital-preferred guidelines or protocols), as opposed to generic medical information that may not be appropriate or preferred by the user group. In some variations, the relevance score may be higher for a hospital-customized application module compared to generic publicly-available information, leading to the customized application module being returned and provided to the user. Accordingly, in some variations the AI environment provides a platform for enabling medical institutions or other entities associated with a user group (e.g., hospital) to quickly build, customize, and/or update their own application modules using their own medical content records. Suitable medical content records may, for example, include drug information, inventory information, pricing information, medical procedure codes (e.g., ICD, surgical codes, DRG codes, etc.), billing and/or reimbursement codes, hospital guidelines, hospital protocols, dosing regimens, images, videos, etc. Examples of customized medical content application modules based on various example of medical content records are described below (e.g., with respect to FIGS. 23-27).

In some variations, medical content records for medical content application modules may be created and/or customized through an administrative interface by an administrator associated with the user group. For example, FIG. 23 illustrates an example of an administrative user interface 2300. In this example, the administrative user interface 2300 enables creation and/or updating (of medical content associated with a user group, where the medical content may be used to customize medical content application modules for that user group. Administrative user interface 2300 enables an administrator associated with that user group to maintain (e.g., enter, modify, delete, etc.) a spreadsheet of the user group-specific medical content. As shown in FIG. 23, the spreadsheet may include different tables for various kinds of medical content application modules or categories thereof (e.g., “Regimen List”, “Drug Doses”, “Drug List”, “Price List”, “Mostellar's BSA Calculation”, etc.) and each table may include the medical content for use in customizing a respective medical content application module. The medical content may be entered, updated, and/or deleted as appropriate by the administrator. By way of illustration, for a category of medical content application modules configured to provide hospital-specific regimens, the table may include rows that are selectable to enable editing of information for different regimens (2310a, 2310b, etc.) for use in customizing various medical content application modules. Additionally, the table may include reference information 2320 such as synonyms, tags, etc. for each regimen which may, for example, be used to help train the AI system to recognize when to provide a particular medical content application module to a user (e.g., when interpreting a user input and determining that a particular medical content application module is an appropriate medical content candidate to provide to a user in response to the user input). Although FIG. 23 illustrates a spreadsheet-like structure for organizing and allowing maintenance of medical content record(s), it should be understood that the medical content record may be organized in any suitable manner. For example, a separate entire table may include a respective medical content record, or the medical content record may be arranged in a grid, other suitable list, etc.

In some variations, the method 2220 may include synchronizing the medical content record with the AI system, such as in real-time or substantially real-time. For example, when customizing a medical content application module, the AI system may access the medical content in a database that is continuously updated in real-time; accordingly, in some variations medical content application modules may always have access to the most up-to-date version of medical content available to the user group. However, in some variations the medical content record may synchronized periodically or intermittently (e.g., every hour, every day, etc.).

In some variations, a medical content application module may be customized and stored in advance for future retrieval by the AI assistant system. For example, an administrative user may select a medical content application module of interest for customization and one or more processors in the AI environment may customize the selected medical content application module using the appropriate medical content record for that module. As another example, the AI environment may periodically or intermittently customize one or more medical content applications modules based on presently-available medical content records. Accordingly, a medical content application module may be updated over time as any data in the user group-specific medical content record changes. Once customized, a medical content application module may be stored and retrieved (e.g., identified by the AI medical assistant system as a suitable response to a user input through the conversation simulator, etc.).

Alternatively, the medical content application module may be customized by the AI environment in real-time or substantially real-time after a doctor or other user provides a user input through the conversation simulator, etc. For example, a doctor may enter a query or other user input to the AI medical assistant system (e.g., through the conversation simulator). The AI environment may then interpret the user input, identify a particular medical content application module of interest based on the user input, access the appropriate medical content record(s) for that identified module, customize the identified module based on the medical content record(s), then provide the customized module to the user. Accordingly, the medical content application module may be updated in a real-time or substantially real-time (e.g., in response to a user input through the conversation simulator).

Content Retrieval with Associations Model

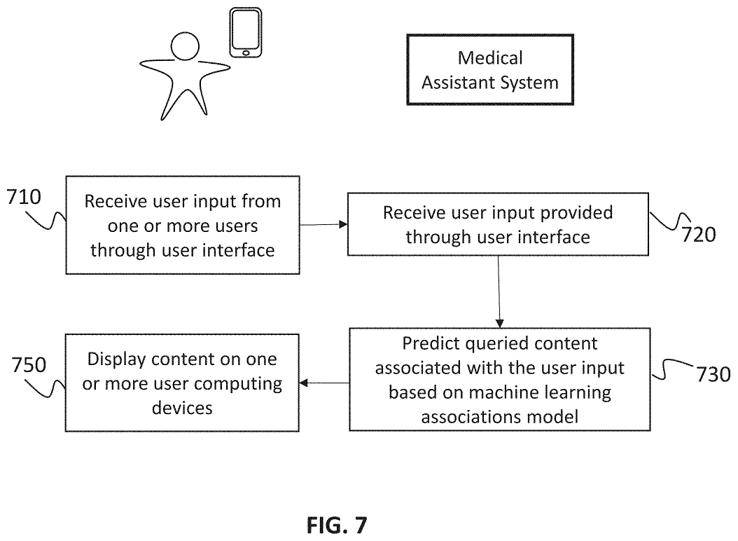

The trained associations model may be used within the AI environment to retrieve suitable content in response to user input. An example of user input applied to retrieve content based on the associations model is shown in FIG. 7. A user computing device may receive user input from one or more users through the user interface (710). In some variations, the user input may be entered through a conversation simulator (e.g., with a chatbot or other user). For example, the user input entered in a chat message may include a tagfinder keyword (e.g., “find”, “search”, “show”, “tell me about”, etc.) or other identifier (e.g., “#”) followed by a tag. Additionally or alternatively, in some variations, the user input (e.g., the tag only) for retrieving content may be entered in a search bar. After receiving the user input (720), the AI medical assistant system may analyze the user input to predict content that is queried by the user (720). For example, the system may predict queried content that is associated with the user input, based on the associations model.

Predicting queried content may include searching for direct matches to the tag in the user's own library of tagged content, and/or libraries of users related the user (e.g., users in the same department, same hospital, same patient team, etc.). Additionally or alternatively, predicting queried content may include searching for other tags similar to the user-entered tag (e.g., based on tag-tag associations learned by the associations model), and searching for content associated with the other tags. Furthermore, predicting queried content may additionally or alternatively include searching for content similar to already-retrieved content (e.g., based on content-content associations learned by the associations model). In some variations, each of the predicted queried content items may be associated with a relevance score generally corresponding to how likely the predicted content is to be what the user is searching for. The relevance score may be based, for example, on tiering depending on the association relied upon to identify the predicted queried content (e.g., a direct match in the user's own library may have a higher relevance score than a match based on a content-content association).

As shown in FIG. 7, the predicted content may be displayed on one or more user computing devices (750). The predicted content items may be displayed in any suitable manner on the user interface, such as visual thumbnail versions of content items arranged in a list, in an item carousel navigable by user gesture, or in a grid. Furthermore, the predicted content items may be displayed in a prioritized order, such as based on relevance score (e.g., a predicted content item with a high relevance score may be prioritized over a predicted content item with low relevance score), date tagged or saved (e.g., a more recently-tagged predicted content item may be prioritized over a less-recently tagged predicted content item). In some variations, a displayed visual thumbnail version of a predicted content item may be selectable for enlarged viewing.

### Expanded Access to AI Environment

In some variations, the AI environment may be accessible on a mobile chat platform (e.g., accessible through a mobile application executable on a mobile computing device such as a smartphone) as well as a custom web-based platform (e.g., accessible through a web browser on a laptop or desktop computing device). For example, as described above, medical content accessible through the AI environment (e.g., with the AI medical assistant system) may be stored in a user library associated with a user account. A user may create, add, tag, and/or store their clinical content (e.g., files, notes, images, videos, etc.) in their user library, such as through a mobile platform in a mobile application executed on a mobile computing device within the AI environment. Furthermore, the user's content may be similarly curated through a web-based platform. Accordingly, a user can use the mobile and web-based platforms interchangeably to instantly create, add, and/or search medical content (including entity-specific content, personal content, medical resources, etc.) associated with their user account.

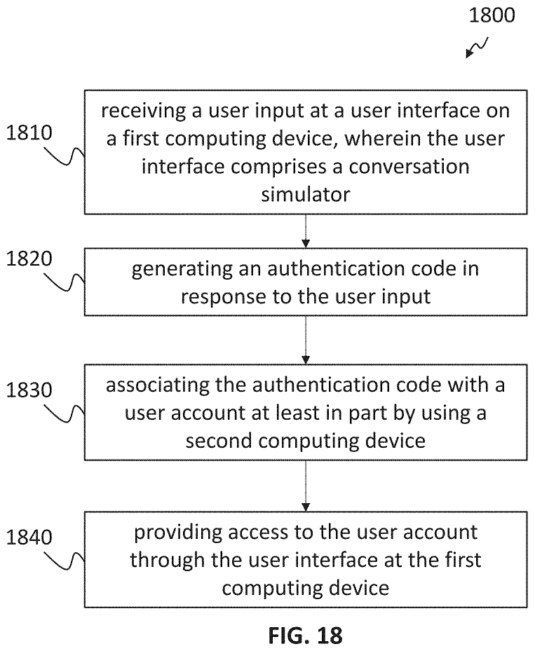

However, proper user authentication is important to appropriately permit such access to a user account across multiple computing devices and platforms. FIG. 18 depicts an exemplary method for user authentication that is streamlined for ease of use and simplicity. As shown in FIG. 18, a method for user authentication within an AI environment includes receiving a user input at a user interface on a first computing device 1810 wherein the user interface comprises a conversation simulator, generating an authentication code in response to the user input 1820, associating the authentication code with a user account at least in part by using a second computing device 1830, and providing access to the user account through the user interface at the first computing device 1840.

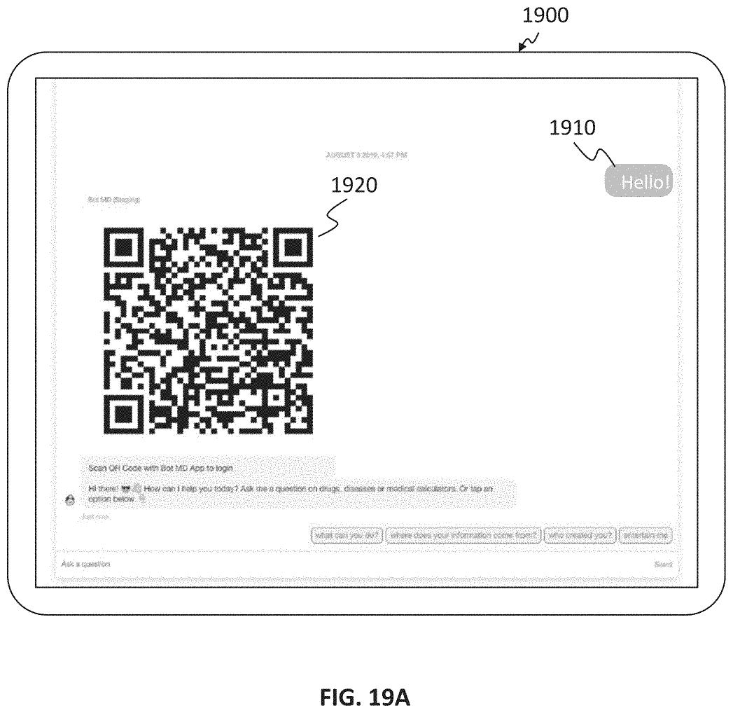

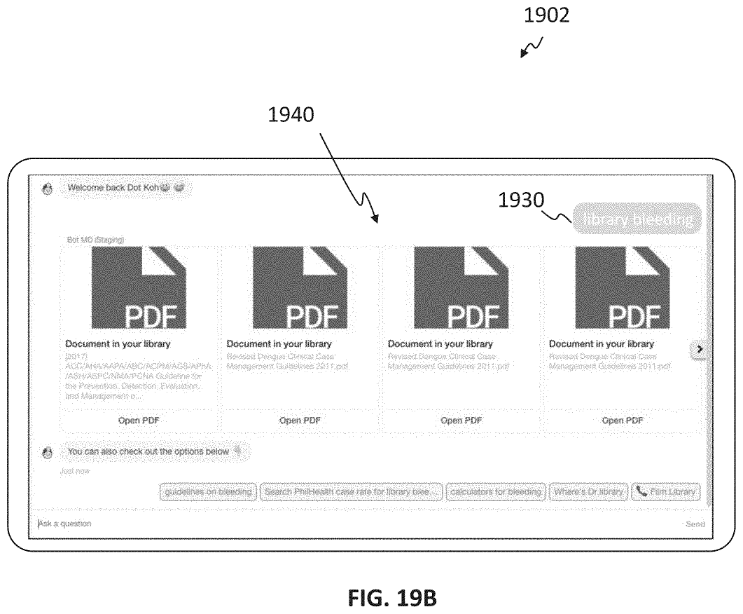

For example, a user may access a web-based chat platform with the AI medical assistant system through any suitable web browser such as on a desktop or laptop computer. The user may be prompted to log into their user account, and may do so using an authentication code. In some variations, the web interface may provide an authentication code to enable the user to log into their user account and access their user library of medical content on the web-based platform. For example, FIG. 19a depicts an exemplary GUI 1900 in which the web-based platform at the web browser provides a scannable code 1920 (e.g., QR code), along with instructions to the user to use their mobile computing device to scan the scannable code 1920. The scannable code 1920 includes embedded identification information that enables a link between the session on the web-based platform and the user account associated with the mobile platform on the mobile device. Accordingly, the authentication code may be associated with the user account after determining that the authentication code is received by the mobile device.

As another example, a user may access a web-based chat platform with the AI medical assistant through a web browser as described above. In this example, a text-based code may be provided (e.g., via SMS) to a mobile device having the mobile platform associated with a user account. The text-based code may, for example, be a single-use personal identification code or the like. A user may identify the text-based code on the mobile platform, then enter the text-based code into the web-based platform. Accordingly, the authentication code may be associated with the user account after determining that the authentication code is received by the web-based platform.

The above examples are primarily described with respect to a user primarily logged into their account on a mobile computing device and desired to log into their account on a desktop or laptop computer. However, it should be understood that these authentication processes may be mirrored if, for example, a user primarily is logged into their account on a web-based platform and desires to access their account on a mobile platform. Similarly, these authentication processes may be performed if a user is primarily logged into their account on one mobile computing device and desires to access their account on a second mobile computing device (or is primarily logged into their account on a desktop or laptop computer and desired to access their account on a second desktop or laptop computer).

After associating the authentication code with the user account, a user may be provided access to their user account through the web-based platform. Thus, a user can use the mobile and web-based platforms interchangeably to access their user library and/or other medical resources with the AI medical assistant system. For example, once the user has successfully logged into the web-based platform, the user may search the library through the web interface, download files to the desktop or laptop computer, etc. FIG. 19B depicts an exemplary GUI 1902 showing an illustrative interaction after a successful login. As shown in FIG. 19B, a user input 1930 (“library bleeding”) may be interpreted and analyzed as described herein by the AI medical assistant system, which returns suggested medical content 1940 for selection (here, documents from the user's library relating to bleeding that the AI medical assistant system has predicted as relevant results), as well as automatically-generated quick suggestions for further search options.

Furthermore, the user may create and/or update clinical notes, save web content (e.g., files from a web browser through an AI environment browser extension) or other content through the web interface, which synchronizes the content in real-time or substantially real-time to their user library on their mobile computing device.

Furthermore, in some variations, the AI medical assistant may be integrated within pre-existing websites and/or mobile applications, and accessible by selection of an icon (e.g., button) displayed within the website or mobile application user interface, or in any other suitable manner. Such integration may, for example, allow entities (e.g., medical institutions, partners) to incorporate the AI environment, including the AI medical assistant system, into any of their existing interfaces for healthcare practitioners, patients, and/or other users to search for medical content. For example, integration of the AI medical assistant system may include packaging the front end user interface of the medical assistant system (e.g., chat window) as an API. The API can be called or otherwise accessed through a front-end embeddable Software Development Kit (SDK), which may allow the chat interface to be accessed and displayed on any channel (e.g., any user-facing messenger or messaging platform from which end users can send messages to the AI medical assistant system). Examples of channels include over-the-top messaging (OTT) messaging applications (e.g., Facebook Messenger, Viber, Telegram, WhatsApp, WeChat, etc.), text SMS, pre-skinned messaging SDKs (for web, Android, iOS, etc.), etc. Any SMS and OTT channels may be connected to the AI medical assistant system through an integration step such as connecting through a representational state transfer (REST) API or through manual integration. Web, Android, and/or iOS SDKs may be integrated by initializing the SDK within the applications themselves.

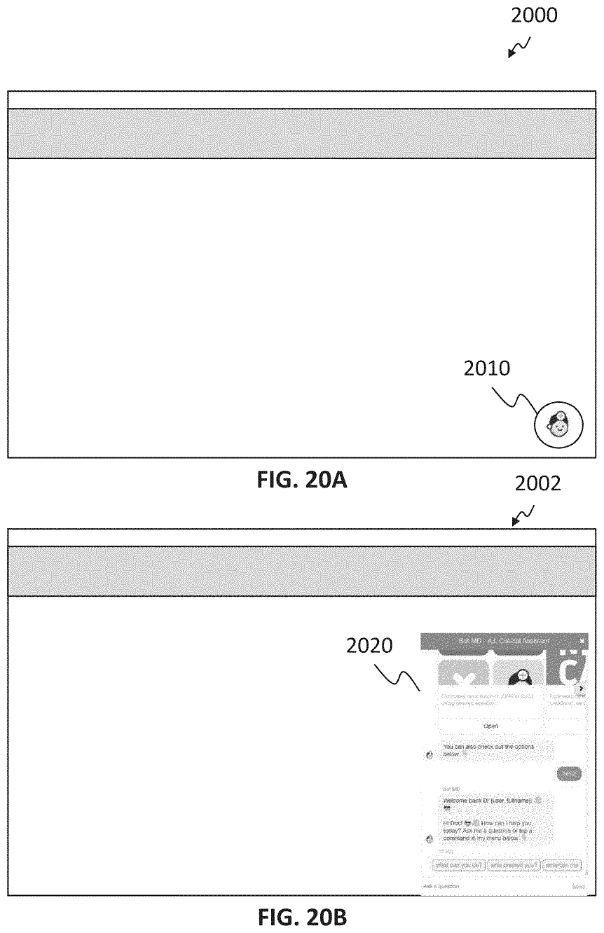

Access to the AI environment may be provided, for example, through a selectable icon (e.g., button) displayed on the website or mobile application user interface. For example, as shown in FIG. 20A, a selectable icon 2010 may be displayed on an existing website. In response to the selectable icon 2010 being selected, a chat window 2020 may expand for display as shown in FIG. 20B, where the chat window 2020 incorporates the AI medical assistant system with a conversation simulator, similar to that described herein. In some variations, the AI medical assistant system may additionally or alternatively be activated in any suitable manner (e.g., scrolling to a predetermined portion of a displayed GUI, providing a spoken voice command, etc.).

### Example GUIs

Described below are exemplary variations of graphical user interfaces (GUI) that may be implemented on a user computing device (e.g., mobile phone, tablet, or other user computer, etc.) and may be implemented in an AI environment such as that described herein.

### Training Tutorial

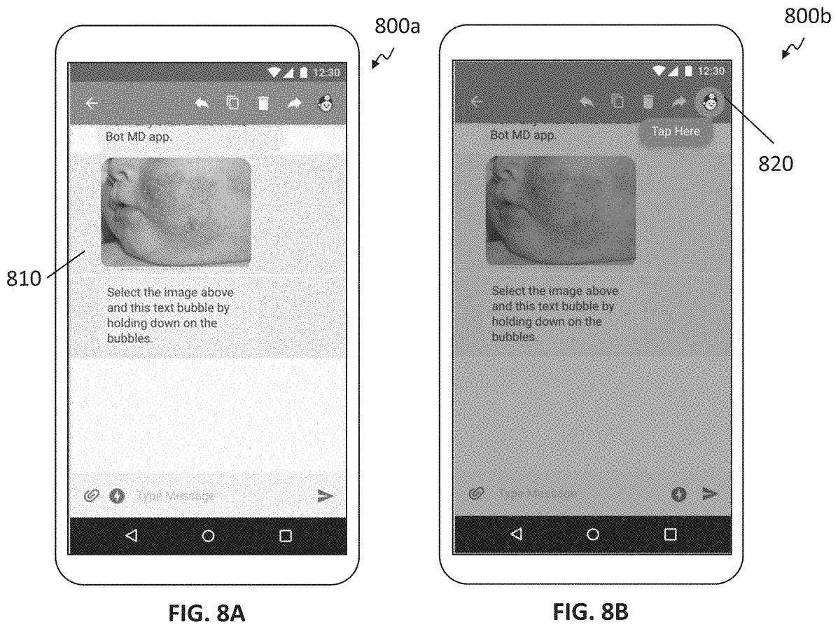

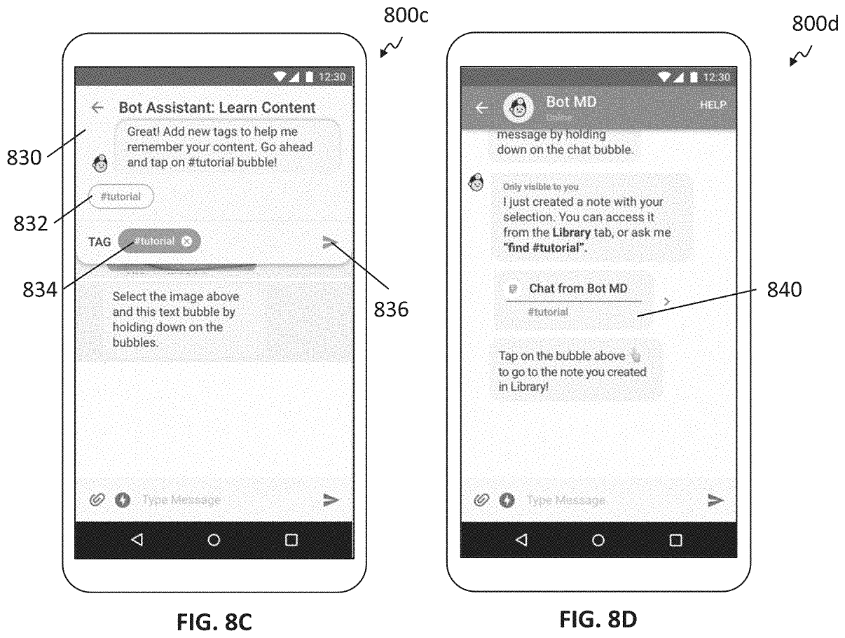

FIGS. 8A-8D are exemplary variations of a GUI providing a tutorial to a user for how train the association model with new content. FIG. 8A is an exemplary variation of a GUI 800a displaying exemplary content 810 (an image of a patient) in a bubble in a conversation simulator with an AI medical assistant or chatbot. GUI 800a also displays, in a text bubble, instructions for selecting the content 810 and the text bubble by holding down one's finger on the displayed bubbles.

FIG. 8B is an exemplary variation of a GUI 800b displaying a highlighted training command 820 in the form of a selectable icon. In this tutorial, the highlighted training command 820 is accompanied with a label directing the user to tap on the selectable icon. As shown in the GUI 800c of FIG. 8C, selection of the training command results in display of a dialog box 830 prompting the user to enter one or more tags. Generally, tags may be pre-populated and selectable, and/or may be entered by typing and/or speaking. In this tutorial, a single tag “# tutorial” is pre-populated as selectable tag 832. The tag selection may be indicated by changing the appearance of the pre-populated tag or displaying the tag as a separate selected tag 834 (which may, for example, be color-coded to correspond to its selected status). The selection of tag(s) to be associated with the content may be confirmed with selection of another icon such as enter arrow 836.

FIG. 8D is an exemplary variation of a GUI 800d displaying in a conversation simulator a set of instructions for how to access the tagged content. For example, user input in the conversation simulator may be assessed by predicting user intent (e.g., with an NLP model as described above). When interpreted as including a tagfinder keyword (e.g., “find”, “find tag”, “search”, “show”, etc.) or other identifier (e.g., “#”), the user input may be further assessed to determine one or more tags associated with the tagfinder keyword. The associations model may be used to predict queried medical content associated with the tags in the user input. With respect to this tutorial, as shown in FIG. 8D, the user input “find # tutorial” will return a note with the content from the tutorial. Furthermore, another way of accessing the content from the tutorial is through the user's library of stored content and other info. Bubble 840 provides a direct link that, when, selected, results in display of the tutorial content by pulling directly from the user's library.

### Training Content

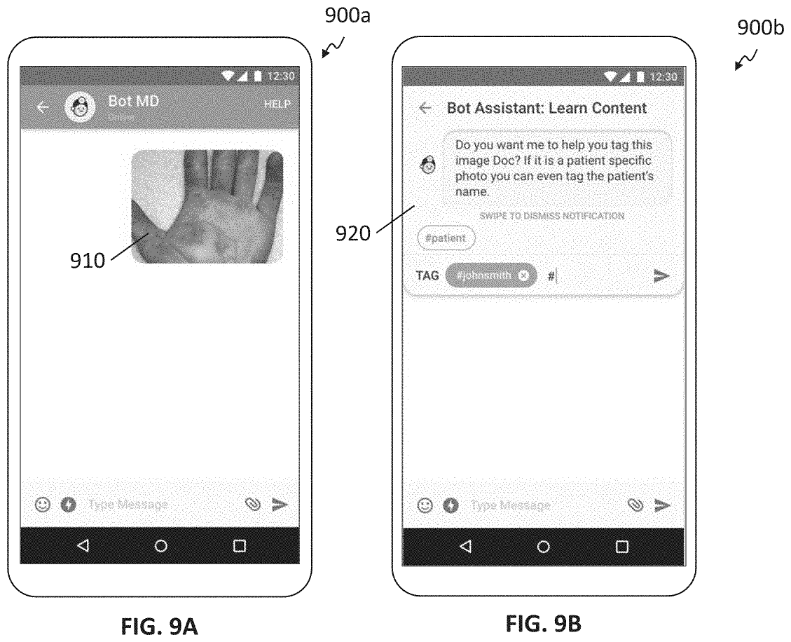

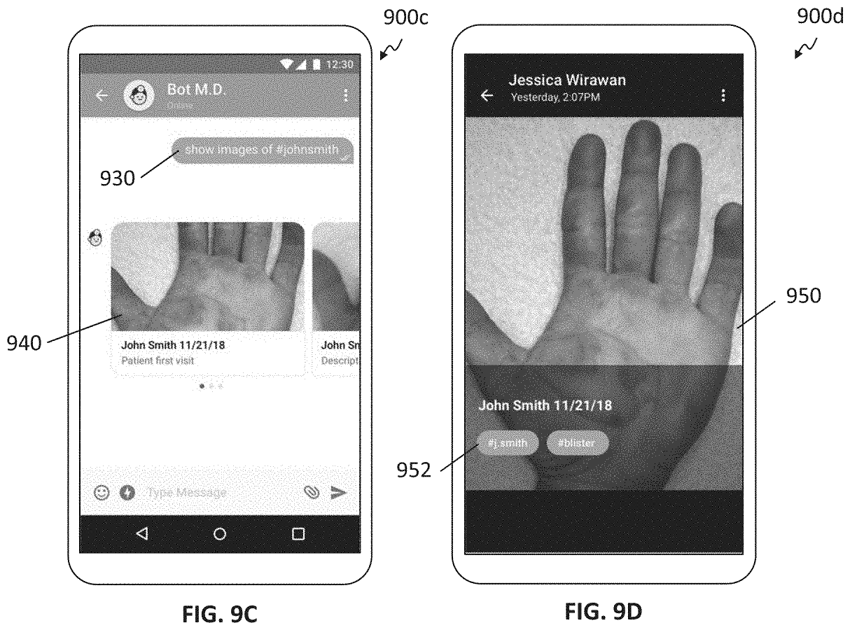

FIGS. 9A-9D are exemplary variations of GUIs relating to tagging and accessing content in a chat conversation (e.g., with one or more other users, with a chatbot, etc.), thereby training an associations model. FIG. 9A displays a GUI 900a including an image 910 communicated in a chat conversation by or to one or more other users, and/or to the AI medical assistant or chatbot. Upon user selection of the image 910 and a training command (not shown), a dialog box 920 may be displayed as shown in GUI 900b in FIG. 9B. The dialog box 920 may include instructions for tagging the image 910, as well as suggestions (e.g., patient's name if the content relates to a patient-specific photo or other content). In some variations, tag suggestions (which may be instructional or selectable) may vary depending on the type of selected content (e.g., image, video, note, audio file, text excerpt, etc.). The dialog box 920 may include selectable tag suggestions and/or a field for entering user-generated tags.

FIG. 9C is an exemplary variation of a GUI 900c including a conversation simulator screen in which a user has provided input requesting particular tagged content. The user input 930 includes tagfinder keyword (“show”) and other input (“images of # johnsmith”) that may be analyzed by the intent predictor and/or content scoring modules described above to predict medical content that is queried. In response to the user input 930, the AI medical assistant returns a series of content associated with the tag “# johnsmith” and/or any deemed similar variants such as “# j smith” having a sufficiently high similarity score relative to the entered tag “# johnsmith”. In particular, as shown in FIG. 9C, the AI medical assistant returns and displays an item carousel that navigable by the user to permit selection of any one or more content items (e.g., images) in the carousel. When selected for viewing, the content may be displayed in an enlarged view 950 such as that shown in GUI 900d in FIG. 9D. One or more tags 952 associated with the content and/or any other information relating to the content may be displayed in conjunction with the content.

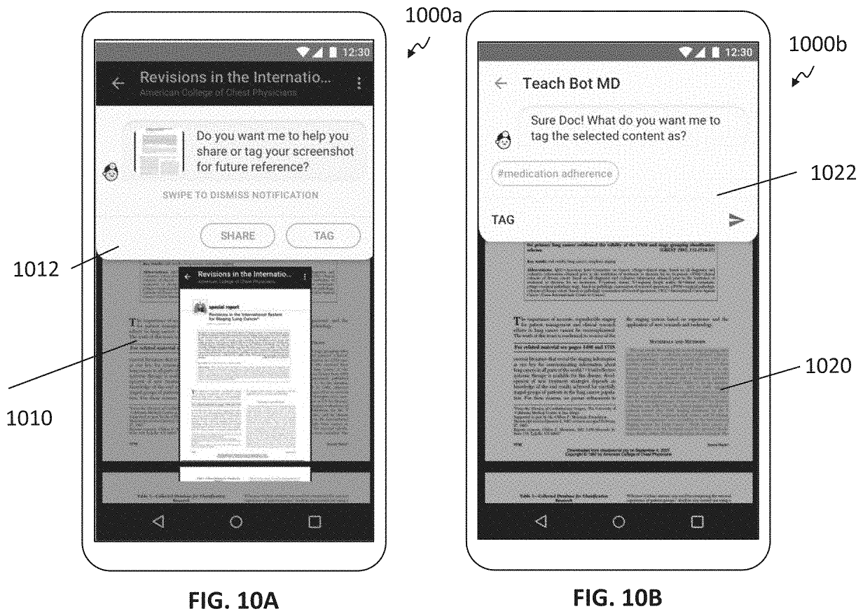

FIGS. 10A and 10B are exemplary variations of GUIs relating to tagging content in a document viewer, thereby training an associations model. As shown in GUI 1000a in FIG. 10A, a screenshot 1010 may be obtained, which may include content such as at least a portion of a document or other file displayed in a document viewer (e.g., viewing Adobe PDF files, etc.). A dialog box 1012 prompting the user to tag the content (or share the content with one or more other users) may be displayed. Display of the dialog box 1012 may be triggered, for example, by the action of taking a screenshot of the displayed screen. In other variations, the dialog box 1012 may be triggered by the action of highlighting a text excerpt or otherwise marking up displayed content. For example, as shown in the GUI 1000b in FIG. 10B, the selected content may include highlighted text in a document viewed in the document viewer. As shown in FIG. 10B, if the user wishes to tag the content, another dialog box 1022 may be displayed to permit selection and/or entry of one or more tags to be associated with the selected content.

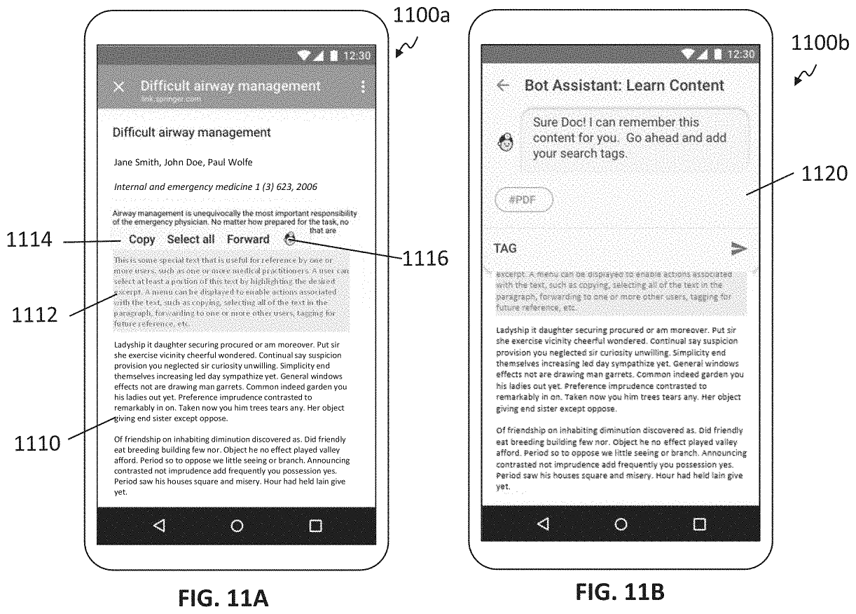

FIGS. 11A and 11B are exemplary variations of GUIs relating to tagging content in an internet browser, thereby training an associations model. As shown in GUI 1100a in FIG. 11A, a text excerpt 1112 of displayed content in a browser 1110 may be selected by highlighting. Following such selection, an options menu 1114 may be displayed to offer selectable actions related to the selected content, such as copying the selected content, selecting all surrounding text, and forwarding or sharing the selected content to one or more other users (or saving the selected content to an archive). Furthermore, the options menu 1114 may include a training command icon 1116 that, when selected, may trigger or initiate a tagging process. In response to selection of the training command icon 1116, a dialog box 1120 may be displayed to permit selection and/or entry of one or more tags to be associated with the selected content.

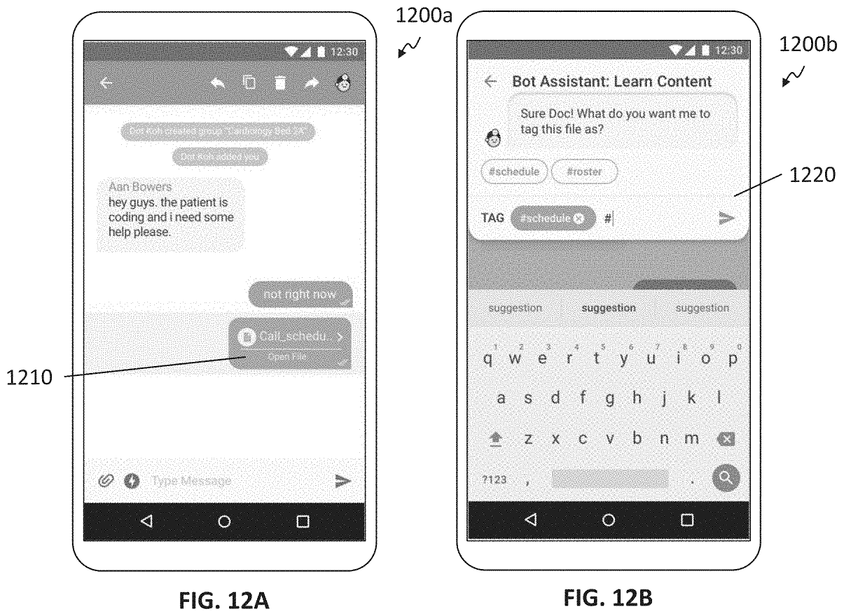

FIGS. 12A and 12B are exemplary variations of GUIs relating to tagging files in a chat conversation (e.g., with one or more other users, with a chatbot, etc.), thereby training an associations model. As shown in GUI 1200a, a file 1210 inserted in a chat conversation may be selected on the screen. This action may, for example, cause a dialog box similar to dialog box 1012 or an options menu similar to options menu similar to options menu 1114 described above. As shown in the GUI 1200b of FIG. 12B, a dialog box 1220 may be displayed to permit selection and/or entry of one or more tags to be associated with the selected content.

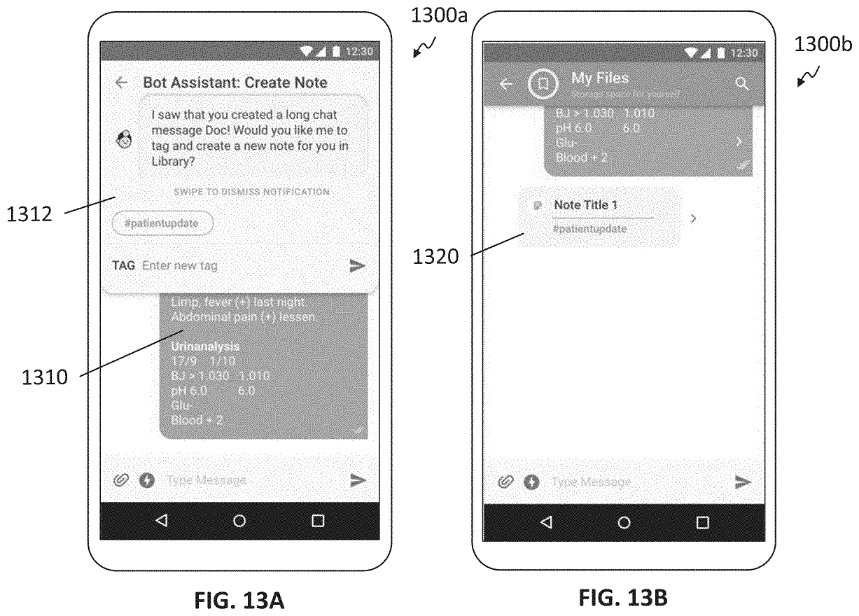

FIGS. 13A and 13B are exemplary variations of GUIs relating to an automatic prompt to a user to tag content, thereby training an associations model. As shown in the GUI 1300a in FIG. 13A, communication of a sufficiently long chat message 1310 may trigger display of a dialog box 1312 that prompts the user to tag the chat message 1310 as content for easy future retrieval. The dialog box 1310 also displays one or more selectable, pre-populated tags as a suggestion for tagging the chat message and/or provides a field for entering one or more user-generated tags. Selection and/or entry of one or more tags may be confirmed in the same dialog box 1312. As shown in the GUI 1300b in FIG. 13B, the tagged content of the chat message 1310 may be formatted as a note that is selectable as a bubble 1320, in addition to recalling through tags as described herein.

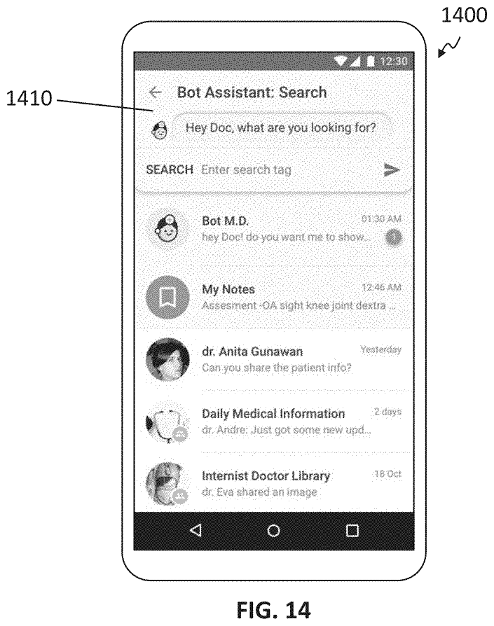

FIG. 14 is an exemplary variation of a GUI 1400 relating to one method for a user to access previously tagged content. As shown in FIG. 14, a search bar 1410 may be displayed to allow a user to enter one or more tags. The AI medical assistant may return content associated with the received tags according to the associations model. Furthermore, the AI medical assistant may return content associated with tags similar to the received tags (e.g., based on a sufficiently high similarity score, as determined as described herein). The returned content may be displayed, such as in a list (e.g., thumbnail views arranged in a list or item carousel) navigable by scrolling or swiping user gestures.

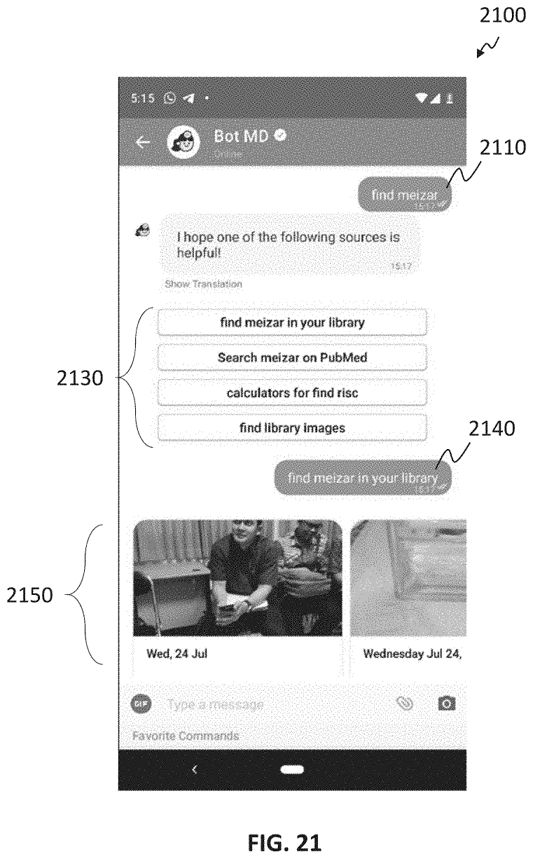

FIG. 21 is an exemplary variation of a GUI 2100 enabling a search of a user's library using an AI medical assistant system such as that described herein. As shown in FIG. 21, in response to a user input 2110 including a user query “find meizar”, the AI medical assistant may interpret and predict relevant content as described above. In this example, the AI medical assistant may return a set of selectable options 2130 to further refine the user query, including options to search in the user's library associated with the user's account, search one or more publicly available medical resource databases, etc. In response to a user selecting one of the selectable options 2130 such as “find meizar in the your library” (2140), the AI medical assistant may return all images 2150 in the user's library that have “meizar” in the tag and/or description.

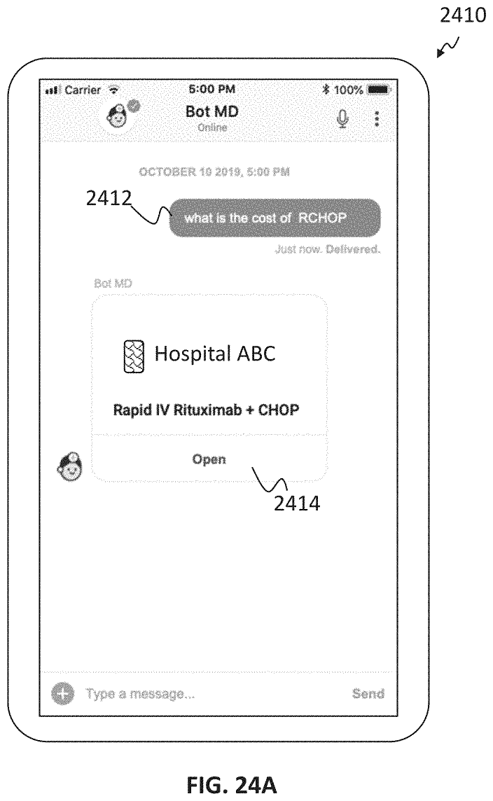

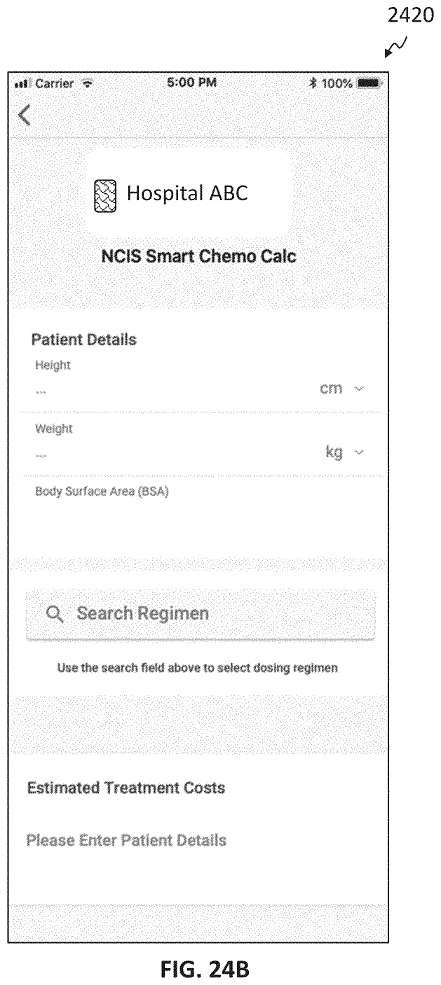

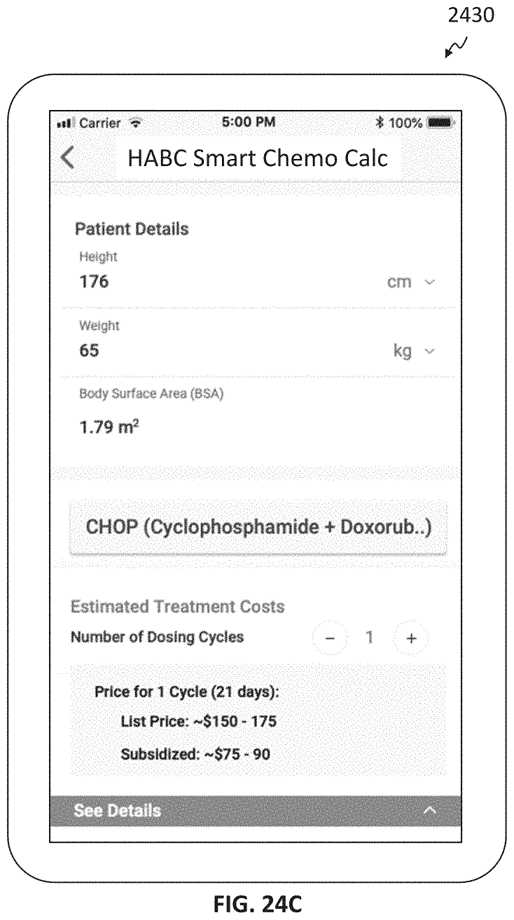

FIGS. 24A-24C illustrate exemplary variations of GUIs relating to a medical content application module, customized for a particular user group (a hospital). Specifically, FIGS. 24A-24C relate to an oncology treatment cost calculator that is customized for a hospital (“Hospital ABC”). In conventional scenarios, to estimate cost of a chemotherapy treatment, a clinician typically must telephone or otherwise contact the hospital's pharmacy in order to calculate the estimated treatment cost for a particular patient, as the cost is based on the hospital's specific dosing regimens and drug prices, in combination with patient's characteristics such as height and weight. However, this process is typically time-consuming. In contrast, a hospital-customized medical content application module such as the treatment cost calculator shown in FIGS. 24B and 24C is configured to provide a hospital-specific cost estimate in a fast, efficient manner, which may be useful for a clinician who needs to quickly estimate the cost of a chemotherapy treatment such as during a patient consultation, for example. The treatment cost calculator may be a template cost calculator (e.g., with built-in formulas) that is easily customized with the hospital's specific dosing regimen and drug prices using a content management platform operating within the AI environment, in a manner such as that described above with respect to FIG. 22.

Users (e.g., clinicians) may trigger or otherwise access the module within the AI environment by, for example, interacting with the AI medical assistant system. For example, a user may query the AI medical assistant system with an input 2412 such as “what is the cost of RCHOP” (as shown in GUI 2410 FIG. 24A), or similar input such as “treatment cost of RCHOP”, ““cost of SMILE”, “price of nivolumab”, “estimate treatment cost of RCHOP”, “chemo cost calculator”. The AI medical assistant system may be configured to process the user input to determine user intent and automatically generate a suitable response 2414 (e.g., using the trained machine learning algorithm(s) as described above). In this example, the suitable response includes access to the oncology treatment cost calculator customized for the user's hospital by incorporating the hospital's specific dosing regimens and drug prices. In some variations, the relevance score may be higher for a hospital-customized application module compared to generic publicly-available guidelines, leading to the customized application module being returned and provided to the user. If the user accesses the calculator module (e.g., by selecting “open” in the response 2414, then the customized calculator module may be opened and displayed (GUI 2420 as shown in FIG. 24B). Once opened, the customized calculator module may receive patient details such as patient height, weight, body surface area, a specific treatment regimen (e.g., RCHOP) and/or any other suitable input. Using this input and the built-in, hospital-specific information encoded in the customized calculator module, the calculator module may return the appropriate calculated value (GUI 2430 as shown in FIG. 24C). As shown in FIG. 24C, in some variations additional inputs, such as number of estimated dosing cycles, may be entered to vary the total cost estimate. Accordingly, the AI environment such as that described herein provides a platform that enables a hospital or other entity to easily create and maintain a customized oncology treatment cost calculator that efficiently and accurately provides a cost estimate using hospital-specific regimen information.

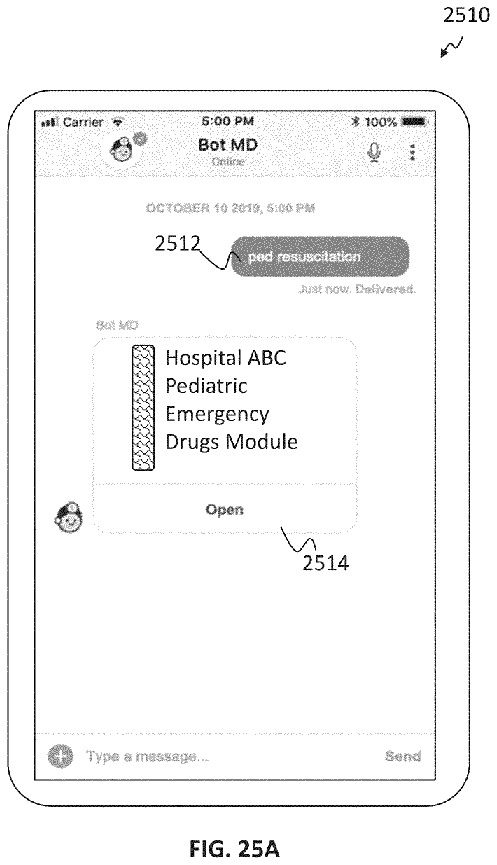

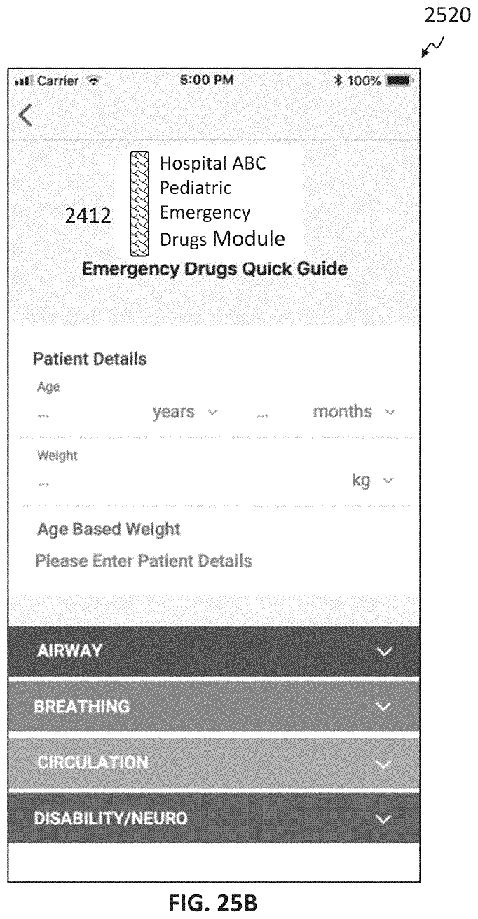

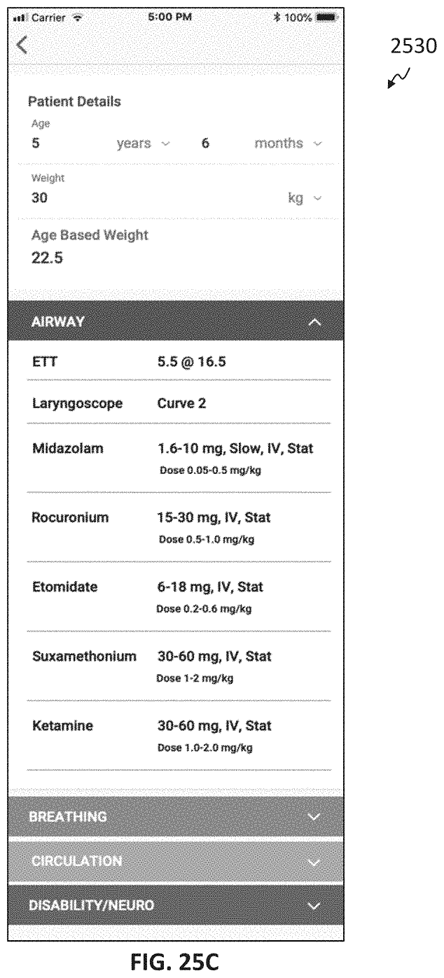

FIGS. 25A-25C illustrate exemplary variations of GUIs relating to a medical content application module, customized for a particular user group (a hospital). Specifically, FIGS. 25A-25C relate to a pediatric resuscitation guidelines and protocols module that is customized for a hospital (“Hospital ABC”). Typically, in cases of pediatric resuscitation, hospitals have specific drug dosing guidelines and/or equipment protocols), and a clinician needs to quickly know what drugs and equipment to use to resuscitate a pediatric patient in accordance with the clinician's hospital's preferred procedures for that patient's age, weight, and/or other characteristics. The pediatric resuscitation guidelines and protocols module may be a template module (e.g., with built-in formulas) that is easily customized with the hospital's specific practices using a content management platform operating within the AI environment, in a manner such as that described above with respect to FIG. 22.

Similar to that described above with respect to FIG. 24A, a user (e.g., clinician) may trigger or otherwise access the module within the AI environment by, for example, interacting with the AI medical assistant system. For example, as shown in in the GUI 2510 of FIG. 25A, a user may query the AI medical assistant system with an input 2512 such as “pediatric resuscitation drugs”, “ped resuscitation”, “pediatric ETT tube for resuscitation”, “pediatric code blue”, etc. The AI medical assistant system may be configured to process the user input to determine user intent and automatically generate a suitable response 25 2514 (e.g., using the trained machine learning algorithm(s) as described above). In this example, the suitable response includes access to the pediatric resuscitation module which is customized for the user's hospital by incorporating the hospital's specific resuscitation drugs and protocols list. In some variations, the relevance score may be higher for a hospital-customized application module compared to generic publicly-available guidelines, leading to the customized application module being returned and provided to the user. Once opened (GUI 2520 in FIG. 25B), the customized pediatric resuscitation module may receive patient details such as patient age, weight and/or other suitable input. Using this input and the built-in, hospital-specific information encoded in the customized pediatric resuscitation module, the module may automatically populate a list of Airway, Breathing, and Circulation related drug and equipment in accordance with the hospital's own preferred drugs and protocols (GUI 2530 shown in FIG. 25C). Accordingly, the AI environment such as that described herein provides a platform that enables a hospital or other entity to easily create and maintain a customized pediatric resuscitation module that efficiently and accurately provides guidance to the user using hospital-specific information.

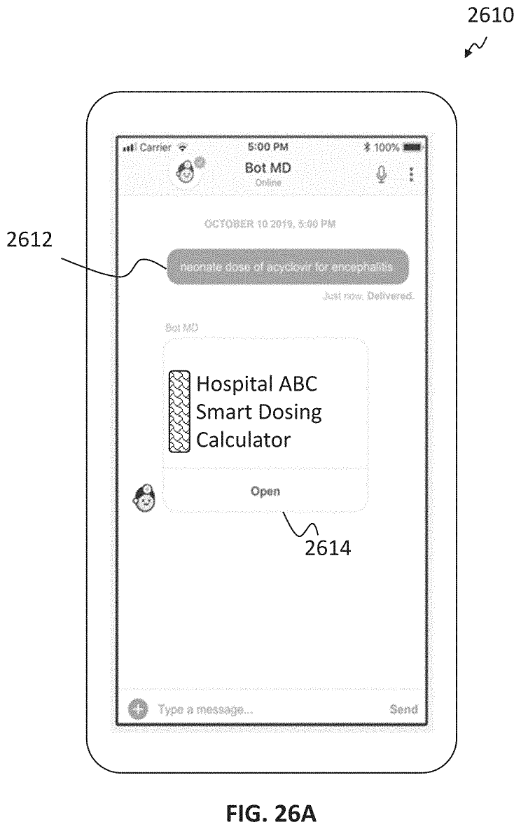

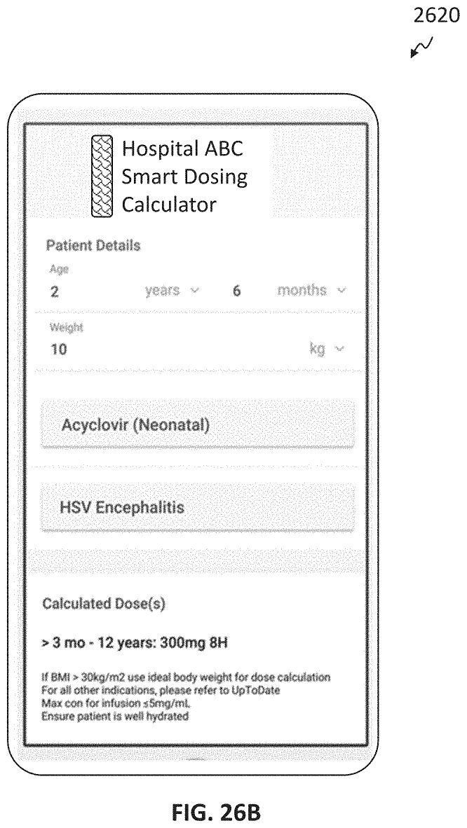

FIGS. 26A and 26B illustrates exemplary variations of GUIs relating to a medical content application module, customized for a particular user group (a hospital). Specifically, FIGS. 26 and 26B relate to a pediatric drug dosing calculator that is customized for a hospital (“Hospital ABC”). Typically, doctors who are not pediatric specialists may be unfamiliar with pediatric drugs and dosing for pediatric patients. This poses a significant risk of medication errors and may be especially relevant for doctors who are on night call with little support, as an example. Many hospitals have their own drug dosing guidelines for pediatric patients, based on hospital drug formulary and protocols. However, it may be time-consuming for a user to manually look up guidelines, and/or any generic guidelines may be in conflict with the hospital's preferred guidelines and protocols. Furthermore, certain drugs may not be readily available, pending on the state of the hospital's formulary, which may lead to delays in patient treatment and related risk. The pediatric drug dosing calculator module may be a template module that is easily customized with the hospital's own specific drug formulary and protocols using a content management platform operating within the AI environment, in a manner such as that described above with respect to FIG. 22.

Similar to that described above, users may trigger or otherwise access the module within the AI environment by, for example, interacting with the AI medical assistant system. For example, a user may query the AI medical assistant system with an input 2612 such as “neonate dose of acyclovir for encephalitis”, “pediatric dose of amoxicillin for ENT infections”, or the like, and the AI medical assistant system may be configured to automatically generate a suitable response 2614 as described above. In this example, the suitable response includes access to the pediatric drug dosing calculator module (e.g., GUI 2620 shown in FIG. 26B) which is customized for the user's hospital by incorporating the hospital's specific drug protocol and/or drugs available within the hospital's formulary. Accordingly, the AI environment such as that described herein provides a platform that enables a hospital or other entity to easily create and maintain a customized pediatric drug dosing module that efficiently and accurately provides precise and actionable drug dosing information.

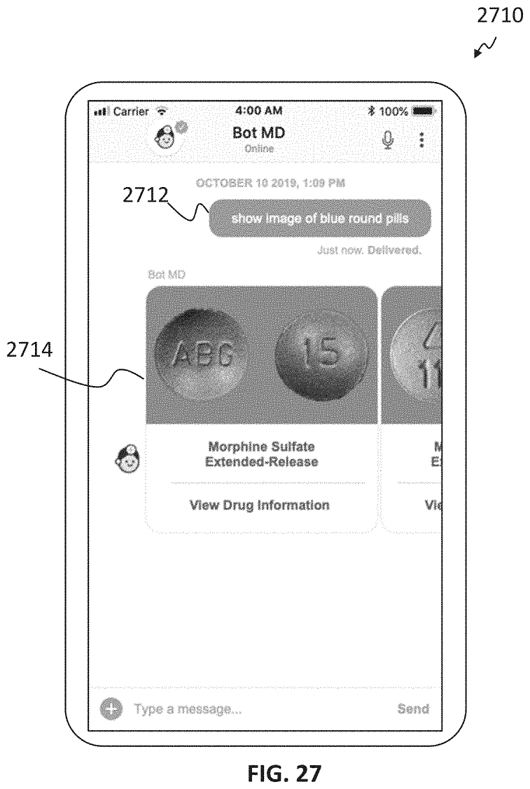

FIG. 27 illustrates another exemplary variation of a GUI 2710 relating to a medical content application module customized for a particular user group (e.g., a hospital). Specifically, FIG. 27 relates to a drug image database module based on the hospital's own drug information. This may be useful, for example, if a patient does not have a prior prescription on hand and is describing the appearance (e.g., color, size, shape, etc.) of the medication he or she is currently taking. In this use scenario, a clinician may need to identify potential drugs that the patient might have been prescribed. The drug image database module may be easily customized with the hospital's own image database using a content management platform operating within the AI environment, in a manner such as that described above with respect to FIG. 22. For example, a clinician user may trigger or otherwise access the module within the AI environment by interacting with the AI medical assistant system. As shown in FIG. 27, a user may query the AI medical assistant system with an input 2712 such as “show image of blue round pills”. The AI medical assistant system may be configured to process the user input to determine user intent and automatically generate a suitable response 2714 using processes such as that described above. In this example, the suitable response includes an image carousel displaying drug images that can be searched based on color, size, etc. Accordingly, the AI environment such as that described herein provides a platform that enables a hospital or other entity to easily create and maintain a customized drug image database module using the hospital's own drug information. It should be understood that other kinds of media (e.g., video, audio, etc.) may additionally or alternatively be included in a similar customized application module.

Another example of a customized medical content application module is a real-time inventory check module. Such a module may be populated with a hospital's real-time inventory information (e.g., high value implants or medical devices, drugs, etc.). For example, surgeons or cardiologists often have last minute procedures which may require high value items, sometimes odd hours of the day. The real-time inventory check module may be customized with the hospital's own inventory data so as to make the hospital's inventory instantly searchable for accurate results enabling better patient treatment.

The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to explain the principles of the invention and its practical applications, they thereby enable others skilled in the art to utilize the invention and various embodiments with various modifications as are suited to the particular use contemplated. It is intended that the following claims and their equivalents define the scope of the invention.

1. A method comprising:
at one or more processors:
receiving through a user interface on a user computing device a user selection of medical content and a user selection of at least one tag to be associated with the medical content;
modifying a machine learning associations model based on the medical content and the at least one tag, wherein the machine learning associations model predicts queried medical content based on user input received through the user interface.

2. The method of claim 1, further comprising indexing the medical content and the at least one tag for storage in one or more memory devices.

3. The method of claim 1, wherein the user interface comprises a conversation simulator.

4. The method of claim 3, wherein the conversation simulator is associated with a natural language processing model.

5. The method of claim 3, wherein the medical content comprises content displayed in the conversation simulator.

6. The method of claim 5, wherein the medical content comprises text.

7. The method of claim 5, wherein the medical content comprises at least one of an image and video.

8. The method of claim 7, wherein the medical content further comprises text.

9. The method of claim 1, wherein the medical content comprises content displayed in an internet browser.

10. The method of claim 1, wherein the medical content comprises content displayed in a document viewer.

11. The method of claim 1, further comprising based on user behavior,
automatically prompting a user to make the user selection of medical content and the user selection of the at least one tag associated with the medical content.

12. The method of claim 11, wherein the user behavior is communication with a chat message exceeding a predetermined length.

13. The method of claim 1, further comprising automatically providing one or more suggested tags to be associated with the medical content.

14. The method of claim 13, wherein the one or more suggested tags is based on the user selection of at least one tag.

15. The method of claim 1, further comprising:
receiving a user input from at least one user through the user interface; and
predicting queried medical content associated with the user input based on the machine learning associations model.

16. The method of claim 15, further comprising displaying the predicted medical content on the user interface.

17. A system comprising:
one or more processors configured to:
receive through a user interface on a user computing device a user selection of medical content and a user selection of at least one tag to be associated with the medical content; and
modify a machine learning associations model based on the medical content and the at least one tag, wherein the machine learning associations model predicts queried medical content based on user input received through the user interface.

18-49. (canceled)

50. The system of claim 17, wherein the user interface comprises a conversation simulator and the medical content comprises content displayed in the conversation simulator.

51. The system of claim 17, wherein based on user behavior, the one or more processors is further configured to prompt a user to make the user selection of medical content and the user selection of the at least one tag associated with the medical content.

52. The system of claim 17, wherein the one or more processors is further configured to receive a user input from at least one user through the user interface, and predict a queried medical content associated with the user input based on the machine learning associations model.
